SEQUENCE LISTING (40 sequences)

SEQ ID NO: 1 - DNA, 22 bp, H. sapiens
cacacacacacacagggccgct

SEQ ID NO: 2 - DNA, 22 bp, H. sapiens
gaaattaaccctcactaaaggg

SEQ ID NO: 3 - DNA, 22 bp, H. sapiens
gtaatacgactcactatagggc

SEQ ID NO: 4 - DNA, 21 bp, H. sapiens
ggtcacgcatctcattcagga

SEQ ID NO: 5 - DNA, 19 bp, H. sapiens
acgtggagatggatgctgc

SEQ ID NO: 6 - DNA, 20 bp, H. sapiens
cggctcaggtccacgccagg

SEQ ID NO: 7 - DNA, 18 bp, H. sapiens
aggcccaaggccagtacc

SEQ ID NO: 8 - DNA, 20 bp, H. sapiens
aggagctgggactggtttga

SEQ ID NO: 9 - DNA, 23 bp, H. sapiens
cctccccatctgcacaccctgtg

SEQ ID NO: 10 - DNA, 19 bp, H. sapiens
atgtgcacgatggcaaggt

SEQ ID NO: 11 - DNA, 20 bp, H. sapiens
ggcagcctcagttcttggtg

SEQ ID NO: 12 - DNA, 24 bp, H. sapiens
tgtccacccacgagcaggtccttc

SEQ ID NO: 13 - DNA, 21 bp, H. sapiens
aacagctaagccaagccactg

SEQ ID NO: 14 - DNA, 20 bp, H. sapiens
tcctcaggacccacaccatt

SEQ ID NO: 15 - DNA, 23 bp, H. sapiens
tgctgccaccaaccacaccactg

SEQ ID NO: 16 - DNA, 26 bp, H. sapiens
tttctttcctccaggtagagttttct

SEQ ID NO: 17 - DNA, 24 bp, H. sapiens
aacgattccaacatcacttctgtg

SEQ ID NO: 18 - DNA, 20 bp, H. sapiens
ccagaggccttgggaaacat

SEQ ID NO: 19 - DNA, 20 bp, H. sapiens
tccgtatgctgctgcagatt

SEQ ID NO: 20 - DNA, 25 bp, H. sapiens
aagaagctctccaaccgtctcgccc

SEQ ID NO: 21 - DNA, 20 bp, H. sapiens
cctgctggtggtggacaaat

SEQ ID NO: 22 - DNA, 19 bp, H. sapiens
gctgcggcccttgttattc

SEQ ID NO: 23 - DNA, 26 bp, H. sapiens
cgacgaacctctgagcatcctggtga

SEQ ID NO: 24 - DNA, 24 bp, H. sapiens
gcaagcagtcagaccaaaatacct

SEQ ID NO: 25 - DNA, 24 bp, H. sapiens
ctagccagtcaattaggatggctc

SEQ ID NO: 26 - DNA, 27 bp, H. sapiens
ctgggtcgggaagtcactggaaacatg

SEQ ID NO: 27 - DNA, 19 bp, H. sapiens
tgctgacttcggctggtct

SEQ ID NO: 28 - DNA, 21 bp, H. sapiens
agggtgccacacattgtcttc

SEQ ID NO: 29 - DNA, 20 bp, H. sapiens
tgcatgcgccctccctgagg

SEQ ID NO: 30 - DNA, 20 bp, H. sapiens
atagccactgcttcgccaga

SEQ ID NO: 31 - DNA, 19 bp, H. sapiens
ctgtgcaggcttaggagcg

SEQ ID NO: 32 - DNA, 21 bp, H. sapiens
aacccagcgccaccatccacg

SEQ ID NO: 33 - DNA, 20 bp, H. sapiens
gggaaatcgtgcgtgacatt

SEQ ID NO: 34 - DNA, 20 bp, H. sapiens
tggccatctcttgctcgaag

SEQ ID NO: 35 - DNA, 26 bp, H. sapiens
aggagaagctgtgctacgtcgccctg

SEQ ID NO: 36 - DNA, 36 bp, H. sapiens
gagagtgagtgatcatgttttttttttttttttttv

SEQ ID NO: 37 - DNA, 24 bp, H. sapiens
agcactctccagcctctcaccgca

SEQ ID NO: 38 - DNA, 12 bp, H. sapiens
gatctgcggtga

SEQ ID NO: 39 - DNA, 28 bp, H. sapiens
agcactctccagcctctcaccgcacatg

SEQ ID NO: 40 - DNA, 8 bp, H. sapiens
tgcggtga

(19) United States
(12) Patent Application Publication
(10) Pub. No.: US 2006/0154275 A1
(43) Pub. Date: Jul. 13, 2006

(54) Regulated genes in cervical cancer

(75) Inventors: Gregory D. Sgarlato, Stanford, CA (US); Catharine Eastman, Palo Alto, CA (US); Howard H. Sussman, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 11/293,598

(22) Filed: Dec. 2, 2005

Related U.S. Application Data
(60) Provisional application No. 60/632,766, filed on Dec. 2, 2004.

(51) Int. Cl. C12Q 1/68; G01N 33/574; C12P 19/34

(52) U.S. Cl. 435/6; 435/7.23; 435/91.2

(57) ABSTRACT

Polynucleotides, as well as polypeptides encoded thereby, that are differentially expressed in SCCC cells are provided. The polynucleotides find use in diagnosis of cancer, and classification of cancer cells according to expression profiles. The methods are useful for detecting cervical cancer cells, facilitating diagnosis of cervical cancer and the severity of the cancer (e.g., tumor grade, tumor burden, and the like) in a subject, facilitating a determination of the prognosis of a subject, and assessing the responsiveness of the subject to therapy.

### BACKGROUND OF THE INVENTION

Cervical cancer is the second most common cancer diagnosis in women and is linked to high-risk human papillomavirus infection 99.7% of the time. Currently, 12,000 new cases of invasive cervical cancer are diagnosed in US women annually, resulting in 5,000 deaths each year. Furthermore, there are approximately 400,000 cases of cervical cancer and close to 200,000 deaths annually worldwide. Human papillomaviruses (HPVs) are one of the most common causes of sexually transmitted disease in the world. Overall, 50-75% of sexually active men and women acquire genital HPV infections at some point in their lives. An estimated 5.5 million people become infected with HPV each year in the US alone, and at least 20 million are currently infected. The more than 100 different isolates of HPV have been broadly subdivided into high-risk and low-risk subtypes based on their association with cervical carcinomas or with benign cervical lesions or dysplasias.

Squamous cell carcinoma of the cervix (SCCC) is by far the most common histological type of cervical cancer. The Pap test, based upon cytological examination of vaginal exfoliated cells, has reduced the incidence and mortality of cervical cancer by 60-70% where it has been used in routine screening programs. However, where no Pap screening programs are in place or where a population does not participate in screening programs, the incidence and mortality of the disease remains high.

A limitation of the Pap test is that it is morphologically based, and the accuracy can be problematic because of pre-analytical processing and interpretive errors. There is inter-observer variation in the reading and classifying of the cytological smears. Molecular-based testing for high-risk human papillomavirus (HPV) strains is mostly performed when Pap tests are inconclusive and is generally used in conjunction with liquid based cytological methods. These tests are still being investigated in large studies to further determine their usefulness.

Current guidelines for managing patients with atypical squamous cells call for assigning these cases into Pap subcategories that distinguish the cases that have a high risk for invasive carcinoma (ASC-H) (HSIL) from the cases of undetermined significance (ASC-US). A molecular test based upon multiple diagnostic markers that are associated with the cancer phenotype potentially could identify SCCC with higher specificity than currently available tests. Furthermore, the identification of a subset of those expressed in SCCC would be helpful in subcategory assignment.

Identification of polynucleotides that correspond to genes that are differentially expressed in cancerous, pre-cancerous, or low metastatic potential cells relative to normal cells of the same tissue type, provides the basis for diagnostic tools, facilitates drug discovery by providing for targets for candidate agents, and further serves to identify therapeutic targets for cancer therapies that are more tailored for the type of cancer to be treated. Early disease diagnosis is of central importance to halting disease progression, and reducing morbidity. The product of a differentially expressed gene can be the basis for screening assays to identify chemotherapeutic agents that modulate its activity (e.g. its expression, biological activity, and the like)

Analysis of a patient sample to identify the gene products that are differentially expressed, and administration of therapeutic agent(s) designed to modulate the activity of those differentially expressed gene products, provides the basis for more specific, rational cancer therapy that may result in diminished adverse side effects relative to conventional therapies. Furthermore, confirmation that a tumor poses less risk to the patient (e.g., that the tumor is benign) can avoid unnecessary therapies. In short, identification of genes and the encoded gene products that are differentially expressed in cancerous cells can provide the basis of therapeutics, diagnostics, prognostics, therametrics, and the like.

The present invention identifies genes that are transcriptionally upregulated in SCCC. The identification of these genes provides insight into the understanding of the biology of SCCC, and the genes identified have use in diagnosis.

### SUMMARY OF THE INVENTION

The present invention provides methods and compositions useful in detection of cervical cancer cells, identification of agents that modulate the phenotype of cervical cancer, and identification of therapeutic targets for chemotherapy. More specifically, the invention provides polynucleotides, as well as polypeptides encoded thereby, that are differentially expressed in cervical cancer cells, particularly squamous cell carcinoma of the cervix (SCCC). Also provided are antibodies that specifically bind the encoded polypeptides. These polynucleotides, polypeptides and antibodies are useful in a variety of diagnostic, therapeutic, and drug discovery methods. In some embodiments, a polynucleotide that is differentially expressed in SCCC is used in diagnostic assays to detect cervical cancer. In other embodiments, a polynucleotide that is differentially expressed in SCCC, and/or a polypeptide encoded thereby, is itself a target for therapeutic intervention.

In one embodiment of the invention, the invention provides a method for detecting or assessing SCCC. The method involves contacting a test sample obtained from a tissue that is suspected of comprising cervical cancer cells with a probe for detecting a gene product differentially expressed in SCCC. Many embodiments of the invention involve a gene identifiable or comprising a sequence selected from Table 2, Group I, which genes are widely expressed in SCCC patients. In other embodiments of the invention, the sequence is selected from Table 2, group II, which sequences are differentially expressed within SCCC patients, allowing for subtyping and/or staging of the cancer. In specific embodiments, detection of gene expression is by detecting a level of an RNA transcript in the test cell sample. In other specific embodiments detection of expression of the gene is by detecting a level of a polypeptide in a test sample.

In another embodiment of the invention, methods are provided for suppressing or inhibiting a cancerous phenotype of a cancerous cell, the method comprising introducing into a mammalian cell an expression modulatory agent (e.g. an antisense molecule, small molecule, antibody, neutralizing antibody, inhibitory RNA molecule, etc.) to inhibition of expression of a gene identified by a sequence set forth in Table 2 Group I; and/or Group II. Inhibition of expression of the gene inhibits development of a cancerous phenotype in the cell. In specific embodiments, the cancerous phenotype is metastasis, aberrant cellular proliferation relative to a normal cell, or loss of contact inhibition of cell growth.

### BRIEF DESCRIPTION OF THE DRAWINGS

FIG. 1A-E. An example of DNA macroarray analysis. A and B. Duplicate DNA macroarrays hybridized to biotin-labeled normal and disease amplified cDNA respectively from patient VNM105 and exposed to film for 20 minutes. Each pair of dots contains a single gene fragment. Boxes indicate three genes that are present at higher levels in disease than normal, indicating transcriptional upregulation (not all such genes are indicated). Boxed genes from top left, reading down then across: KRT14, NDRG1, NQO1. C and D. Average integrated optical density (intensity) of each pair of dots shown in A and B. *: value is not above background. **: value above background is due exclusively to a blot artifact rather than a true hybridization event. E. Average disease intensity divided by average normal intensity. Gray background indicates a ratio of 1.8 or greater. NC: not calculated (neither normal nor disease intensity for that gene fragment is above background.

FIG. 2. Comparison of results from DNA macroarray and real-time quantitative RT-PCR experiments. Solid circle: transcriptional upregulation detected by both DNA macroarray (disease/normal=2.0 or greater) and real-time quantitative RT-PCR (disease/normal 1.8 or greater). Filled top half circle: transcriptional upregulation detected by DNA macroarray but not real-time quantitative RT-PCR. Filled bottom half circle: transcriptional upregulation detected by real-time quantitative RT-PCR but not DNA macroarray. Empty circle: transcriptional upregulation not detected by DNA macroarray and real-time quantitative RT-PCR. Failure to detect transcriptional upregulation includes failure to detect transcript in normal and disease samples, equal amounts of transcript in normal and disease, and transcriptional downregulation in disease versus normal. Genes are listed in order from those upregulated in the greatest number of patients to least as determined by both DNA macroarray and real-time quantitative RT-PCR (solid circles). Patients are listed in order from those with the greatest number of upregulated genes to least as determined by both DNA macroarray and real-time quantitative RT-PCR.

### DETAILED DESCRIPTION OF THE INVENTION

The present invention identifies polynucleotides, as well as polypeptides encoded thereby, that are differentially expressed in SCCC cells. Methods are provided in which these polynucleotides and polypeptides are used for detecting, assessing, and reducing the growth of cancer cells. The invention finds use in the prevention, treatment, detection or research of cervical cancer.

The present invention provides methods of using the polynucleotides described herein in diagnosis of cancer, and classification of cancer cells according to expression profiles. The methods are useful for detecting cervical cancer cells, facilitating diagnosis of cervical cancer and the severity of the cancer (e.g., tumor grade, tumor burden, and the like) in a subject, facilitating a determination of the prognosis of a subject, and assessing the responsiveness of the subject to therapy. The detection methods of the invention can be conducted in vitro or in vivo, on isolated cells, or in whole tissues or a bodily fluid, e.g., blood, plasma, serum, urine, and the like. Samples of particular interest include cervical tissue, which may be obtained by biopsy, scrape, swab, and the like.

RDA was used to identify the upregulated transcripts in cervical cancer samples. The selected pool of transcripts were then screened by comparative hybridization on DNA macroarrays with amplified cDNA patient samples. RDA subtraction using normal and disease tissues from a single patient reduced the transcriptome complexity and allowed the isolation of key candidates with the screening of relatively few clones. Real-time quantitative RT-PCR was used to confirm the transcriptional upregulation of genes identified by RDA procedure across multiple patients. The validated amplicons may be used in array hybridization and other expression analysis and diagnostic platforms, particularly in cases where the original source material is limiting.

Before the present invention is described, it is to be understood that this invention is not limited to particular embodiments described, as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications and patent applications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a polynucleotide” includes a plurality of such polynucleotides and reference to “the cancer cell” includes reference to one or more cells and equivalents thereof known to those skilled in the art, and so forth.

The publications and applications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Cervical cancer is essentially a sexually transmitted disease. Risk is inversely related to age at first intercourse and directly related to the lifetime number of sexual partners. Risk is also increased for sexual partners of men whose previous partners had cervical cancer. Human papillomavirus (HPV) infection and the development of cervical neoplasia are strongly associated. HPV infection is linked to all grades of cervical intraepithelial neoplasia (CIN) and invasive cervical cancer. Infection with HPV types 16, 18, 31, 33, 35, and 39 increases the risk of neoplasia. However, other factors appear to contribute to malignant transformation. For example, cigarette smoking is associated with an increased risk of CIN and cervical cancer.

Squamous cell carcinoma accounts for 80 to 85% of all cervical cancers. Precursor cells (cervical dysplasia, CIN) develop into invasive cervical cancer over a number of years. CIN grades I, II, and III correspond to mild, moderate, and severe cervical dysplasia. CIN III, which includes severe dysplasia and carcinoma in situ, is unlikely to regress spontaneously and, if untreated, may eventually penetrate the basement membrane, becoming invasive carcinoma. Invasive cervical cancer usually spreads by direct extension into surrounding tissues and the vagina or via the lymphatics to the pelvic and para-aortic lymph nodes drained by the cervix. Hematologic spread is possible.

More than 90% of early asymptomatic cases of CIN can be detected preclinically by cytologic examination of Pap smears obtained directly from the cervix. However, the false-negative rate is 15 to 40%, depending on the patient population and the laboratory. About 50% of patients with cervical cancer have never had a Pap smear or have not had one for >=10 yr. The patients at higher risk for cervical neoplasia are the least likely to be tested regularly. An abnormal Pap smear, i.e. suggesting neoplasia, including dysplasia, CIN, carcinoma in situ, microinvasive carcinoma, or invasive carcinoma, requires further evaluation based on the descriptive diagnosis of the Pap smear and the patient's risk factors.

Suspicious cervical lesions should be biopsied directly. If there is no obvious invasive lesion, colposcopy can be used to identify areas that require biopsy and to localize the lesion. Colposcopy results can be clinically correlated (by assessing characteristic color changes, vascular patterns, and margins) with the results of the Pap smear. If cervical disease is invasive, staging is performed on the basis of the physical examination, with a metastatic survey including cystoscopy, sigmoidoscopy, IV pyelography, chest x-ray, and skeletal x-rays. For early-stage disease (IB or less), chest x-ray is usually the only adjunctive test needed. CT or MRI of the abdomen and pelvis is optional; the results cannot be used to determine the clinical stage.

Invasive squamous cell carcinoma usually remains localized or regional for a considerable time; distant metastases occur late. The 5-yr survival rates are 80 to 90% for stage I, 50 to 65% for stage II, 25 to 35% for stage III, and 0 to 15% for stage IV. Nearly 80% of recurrences manifest within 2 yr. Adverse prognostic factors include lymph node involvement, large tumor size and volume, deep cervical stromal invasion, parametrial invasion, vascular space invasion, and neuroendocrine histology.

As used herein, the terms “a gene that is differentially expressed in a cancer cell,” and “a polynucleotide that is differentially expressed in a cancer cell”, are used interchangeably herein, and generally refer to a polynucleotide that represents or corresponds to a gene that is differentially expressed in a cancerous cell when compared with a cell of the same cell type that is not cancerous, e.g., mRNA is found at levels at least about 25%, at least about 50% to about 75%, at least about 90%, at least about 1.5-fold, at least about 2-fold, at least about 3-fold, at least about 5-fold, at least about 10-fold, or at least about 50-fold or more, different (e.g., higher or lower). The comparison can be made in tissue, for example, if one is using in situ hybridization or another assay method that allows some degree of discrimination among cell types in the tissue. The comparison may also or alternatively be made between cells removed from their tissue source. The term “a polypeptide associated with cancer” refers to a polypeptide encoded by a polynucleotide that is differentially expressed in a cancer cell.

A polynucleotide or sequence that corresponds to, or represents a gene means that at least a portion of a sequence of the polynucleotide is present in the gene or in the nucleic acid gene product (e.g., mRNA or cDNA). A subject nucleic acid may also be “identified” by a polynucleotide if the polynucleotide corresponds to or represents the gene. Genes identified by a polynucleotide may have all or a portion of the identifying sequence wholly present within an exon of a genomic sequence of the gene, or different portions of the sequence of the polynucleotide may be present in different exons (e.g., such that the contiguous polynucleotide sequence is present in an mRNA, either pre- or post-splicing, that is an expression product of the gene). An “identifying sequence” is a minimal fragment of a sequence of contiguous nucleotides that uniquely identifies or defines a polynucleotide sequence or its complement.

The polynucleotide may represent or correspond to a gene that is modified in a cancerous cell relative to a normal cell. The gene in the cancerous cell may contain a deletion, insertion, substitution, or translocation relative to the polynucleotide and may have altered regulatory sequences, or may encode a splice variant gene product, for example. The gene in the cancerous cell may be modified by insertion of an endogenous retrovirus, a transposable element, or other naturally occurring or non-naturally occurring nucleic acid.

Sequences of interest include those set forth in Table 2, group I, which are widely expressed in SCCC, and include the following sequences: CCNB1 (Genbank accession NM_{—}031966); KRT14 (Genbank accession NM_{—}000526); ALDH3A1 (Genbank accession NM_{—}000691); CALML5 (Genbank accession NM_{—}017422); EIF4A1 (Genbank accession NM_{—}001416); HNRPM1 (Genbank accession NM_{—}005968); KARS (Genbank accession NM_{—}005548); KRT16 (Genbank accession NM_{—}005557); NDRG1 (Genbank accession NM_{—}006096 992-1330); OAZ1 (Genbank accession NM_{—}004152); SPINT2 (Genbank accession NM_{—}021102); TKT (Genbank accession NM_{—}001064); ZNF9 (Genbank accession NM_{—}003418); ZWINT (Genbank accession NM_{—}032997); AP2M1 (Genbank accession NM_{—}004068); CBR1 (Genbank accession NM_{—}001757); CES1 (Genbank accession NM_{—}001266); FDX1 (Genbank accession NM_{—}004109); G1P2 (Genbank accession NM_{—}005101); GAPDH (Genbank accession NM_{—}002046); KRT13 (Genbank accession NM_{—}153490); KRT6A (Genbank accession NM_{—}005554); NQO1 (Genbank accession NM_{—}000903); P4HB (Genbank accession NM_{—}000918); PGDH (Genbank accession NM_{—}002631); S100A9 (Genbank accession NM_{—}002965); TALDO1 (Genbank accession NM_{—}006755); 18S rRNA (Genbank accession XO3205); AURKB (Genbank accession NM_{—}004217); CDCA8 (Genbank accession NM_{—}018101); cDNA (Genbank accession DKFZp68602421); FLJ23841 (Genbank accession NM_{—}144589); HM74 (Genbank accession NM_{—}006018); HPV16E7 (Genbank accession AF003020); MGC14799 (Genbank accession NM_{—}032336); MYBL2 (Genbank accession NM_{—}002466); PSMD4 (Genbank accession NM_{—}002810); SPATA11 (Genbank accession NM_{—}032306); TNFS10 (Genbank accession NM_{—}003810); TUBG1 (Genbank accession NM_{—}001070); Yif1p (Genbank accession NM_{—}033557). These sequences are upregulated in a majority of SCCC patient samples.

Sequences of interest also include those set forth in Table 2, group II, which are upregulated in subsets of SCCC, and include the following sequences: AKR1B10 (Genbank accession NM_{—}020299); ARHGAP4 (Genbank accession NM_{—}001666); ASF1B (Genbank accession NM_{—}018154); DTYMK (Genbank accession NM_{—}012145); FLJ10156 (Genbank accession NM_{—}019013); H17 (Genbank accession NM_{—}017547); JFC1 (Genbank accession NM_{—}032872); MCG10911 (Genbank accession NM_{—}032302); MCM2 3′ (Genbank accession NM_{—}004526); novel transcript AY714068ACO2 (Genbank accession NM_{—}001098); cDNA DKFZp434B0425 (Genbank accession AL157459); NEFL (Genbank accession NM_{—}006158); NOD9 (Genbank accession NM_{—}024618); PP3856 (Genbank accession NM_{—}145201); RAPGEFL1 (Genbank accession NM_{—}016339); novel transcript (Genbank accession AY714069); novel transcript AY714070FLJ36635 (Genbank accession AK093954); RHBDF1 (Genbank accession NM_{—}022450); novel transcript AY714071OKL38 (Genbank accession NM_{—}182981).

Further sequences of interest include those set forth in Tables 5 and 6, which represent upregulated and downregulated sequences, respectively.

“Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

“Therapeutic target” refers to a gene or gene product that, upon modulation of its activity (e.g., by modulation of expression, biological activity, and the like), can provide for modulation of the cancerous phenotype.

As used throughout, “modulation” is meant to refer to an increase or a decrease in the indicated phenomenon (e.g., modulation of a biological activity refers to an increase in a biological activity or a decrease in a biological activity).

The invention provides polynucleotides that represent genes that are expressed in human SCCC. These polynucleotides (or polynucleotide fragments) have uses that include, but are not limited to, diagnostic probes and primers as starting materials for probes and primers, as discussed herein. Nucleic acid compositions include fragments and primers, and are at least about 15 bp in length, at least about 30 bp in length, at least about 50 bp in length, at least about 100 bp, at least about 200 bp in length, at least about 300 bp in length, at least about 500 bp in length, at least about 800 bp in length, at least about 1 kb in length, at least about 2.0 kb in length, at least about 3.0 kb in length, at least about 5 kb in length, at least about 10 kb in length, at least about 50 kb in length and are usually less than about 200 kb in length. In some embodiments, a fragment of a polynucleotide is the coding sequence of a polynucleotide. Also included are variants or degenerate variants of a sequence provided herein. In general, a variants of a polynucleotide provided herein have a fragment of sequence identity that is greater than at least about 65%, greater than at least about 70%, greater than at least about 75%, greater than at least about 80%, greater than at least about 85%, or greater than at least about 90%, 95%, 96%, 97%, 98%, 99% or more (i.e. 100%) as compared to an identically sized fragment of a provided sequence. as determined by the Smith-Waterman homology search algorithm as implemented in MPSRCH program (Oxford Molecular). Nucleic acids having sequence similarity can be detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under high stringency conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences under stringent hybridization conditions.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide. mRNA species can also exist with both exons and introns, where the introns may be removed by alternative splicing. Furthermore it should be noted that different species of mRNAs encoded by the same genomic sequence can exist at varying levels in a cell, and detection of these various levels of mRNA species can be indicative of differential expression of the encoded gene product in the cell.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

Probes specific to the polynucleotides described herein can be generated using the polynucleotide sequences disclosed herein. The probes are usually a fragment of a polynucleotide sequences provided herein. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of any one of the polynucleotide sequences provided herein.

The nucleic acid compositions described herein can be used to, for example, produce polypeptides, as probes for the detection of mRNA in biological samples (e.g., extracts of human cells) or cDNA produced from such samples, to generate additional copies of the polynucleotides, to generate ribozymes or antisense oligonucleotides, and as single stranded DNA probes or as triple-strand forming oligonucleotides.

The probes described herein can be used to, for example, determine the presence or absence of any one of the polynucleotide provided herein or variants thereof in a sample. These and other uses are described in more detail below. In one embodiment, the probes are used in an RDA method for analysis of gene expression. In another embodiment, real time PCR analysis is used to analyze gene expression.

The polypeptides contemplated by the invention include those encoded by the disclosed polynucleotides and the genes to which these polynucleotides correspond, as well as nucleic acids that, by virtue of the degeneracy of the genetic code, are not identical in sequence to the disclosed polynucleotides. Further polypeptides contemplated by the invention include polypeptides that are encoded by polynucleotides that hybridize to polynucleotide of the sequence listing. Thus, the invention includes within its scope a polypeptide encoded by a polynucleotide having the sequence of any one of the polynucleotide sequences provided herein, or a variant thereof.

In general, the term “polypeptide” as used herein refers to both the full length polypeptide encoded by the recited polynucleotide, the polypeptide encoded by the gene represented by the recited polynucleotide, as well as portions or fragments thereof. “Polypeptides” also includes variants of the naturally occurring proteins, where such variants are homologous or substantially similar to the naturally occurring protein, and can be of an origin of the same or different species as the naturally occurring protein. In general, variant polypeptides have a sequence that has at least about 80%, usually at least about 90%, and more usually at least about 98% sequence identity with a differentially expressed polypeptide described herein. The variant polypeptides can be naturally or non-naturally glycosylated, i.e., the polypeptide has a glycosylation pattern that differs from the glycosylation pattern found in the corresponding naturally occurring protein.

Fragments of the polypeptides disclosed herein, particularly biologically active fragments and/or fragments corresponding to functional domains, are of interest. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of any one of the polynucleotide sequences provided herein, or a homolog thereof. A fragment “at least 20 aa in length,” for example, is intended to include 20 or more contiguous amino acids from, for example, the polypeptide encoded by a cDNA, in a cDNA clone contained in a deposited library or the complementary stand thereof. In this context “about” includes the particularly recited value or a value larger or smaller by several (5, 4, 3, 2, or 1) amino acids. The protein variants described herein are encoded by polynucleotides that are within the scope of the invention. The genetic code can be used to select the appropriate codons to construct the corresponding variants. The polynucleotides may be used to produce polypeptides, and these polypeptides may be used to produce antibodies by known methods described above and below.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast higher plant, insect, and mammalian cells.

Gene products, including polypeptides, mRNA (particularly mRNAs having distinct secondary and/or tertiary structures), cDNA, or complete gene, can be prepared and used for raising antibodies for experimental, diagnostic, and therapeutic purposes. Antibodies may be used to identify SCCC cells or subtypes. The polynucleotide or related cDNA is expressed as described above, and antibodies are prepared. These antibodies are specific to an epitope on the polypeptide encoded by the polynucleotide, and can precipitate or bind to the corresponding native protein in a cell or tissue preparation or in a cell-free extract of an in vitro expression system.

The antibodies may be utilized for immunophenotyping of cells and biological samples. The translation product of a differentially expressed gene may be useful as a marker. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al. Cell, 96:737-49 (1999)). These techniques allow for the screening of particular populations of cells; in immunohistochemistry of biopsy samples; in detecting the presence of markers shed by cancer cells into the blood and other biologic fluids, and the like.

### Diagnostic Methods

The present invention provides methods of using the polynucleotides described herein in diagnosis of cancer, and classification of cancer cells according to expression profiles. The methods are useful for detecting cancer cells, facilitating diagnosis of cancer and the severity of a cancer (e.g., tumor grade, tumor burden, and the like) in a subject, facilitating a determination of the prognosis of a subject, and assessing the responsiveness of the subject to therapy (e.g., by providing a measure of therapeutic effect through, for example, assessing tumor burden during or following a chemotherapeutic regimen). Detection can be based on detection of a polynucleotide that is differentially expressed in a cancer cell, and/or detection of a polypeptide encoded by a polynucleotide that is differentially expressed in a cancer cell. The detection methods of the invention can be conducted in vitro or in vivo, on isolated cells, or in whole tissues or a bodily fluid, e.g., blood, plasma, serum, urine, and the like).

In general, methods of the invention involving detection of a gene product (e.g., mRNA, cDNA generated from such mRNA, and polypeptides) contact a sample with a probe specific for the gene product of interest. “Probe” as used herein in such methods is meant to refer to a molecule that specifically binds a gene product of interest (e.g., the probe binds to the target gene product with a specificity sufficient to distinguish binding to target over non-specific binding to non-target (background) molecules). “Probes” include, but are not necessarily limited to, nucleic acid probes (e.g., DNA, RNA, modified nucleic acid, and the like), antibodies (e.g., antibodies, antibody fragments that retain binding to a target epitope, single chain antibodies, and the like), or other polypeptide, peptide, or molecule (e.g., receptor ligand) that specifically binds a target gene product of interest.

The probe and sample suspected of having the gene product of interest are contacted under conditions suitable for binding of the probe to the gene product. For example, contacting is generally for a time sufficient to allow binding of the probe to the gene product (e.g., from several minutes to a few hours), and at a temperature and conditions of osmolarity and the like that provide for binding of the probe to the gene product at a level that is sufficiently distinguishable from background binding of the probe (e.g., under conditions that minimize non-specific binding). Suitable conditions for probe-target gene product binding can be readily determined using controls and other techniques available and known to one of ordinary skill in the art.

In some embodiments, methods are provided for a detecting cancer cell by detecting in a cell, a polypeptide encoded by a gene differentially expressed in a cancer cell. Any of a variety of known methods can be used for detection, including, but not limited to, immunoassay, using an antibody specific for the encoded polypeptide, e.g., by enzyme-linked immunosorbent assay (ELISA), radioimmunoassay (RIA), and the like; and functional assays for the encoded polypeptide, e.g., binding activity or enzymatic activity.

For example, an immunofluorescence assay can be easily performed on cells without first isolating the encoded polypeptide. The cells are first fixed onto a solid support, such as a microscope slide or microtiter well. This fixing step can permeabilize the cell membrane. The permeablization of the cell membrane permits the polypeptide-specific probe (e.g, antibody) to bind. Alternatively, where the polypeptide is secreted or membrane-bound, or is otherwise accessible at the cell-surface (e.g., receptors, and other molecule stably-associated with the outer cell membrane or otherwise stably associated with the cell membrane, such permeabilization may not be necessary.

Next, the fixed cells are exposed to an antibody specific for the encoded polypeptide. To increase the sensitivity of the assay, the fixed cells may be further exposed to a second antibody, which is labeled and binds to the first antibody, which is specific for the encoded polypeptide. Typically, the secondary antibody is detectably labeled, e.g., with a fluorescent marker. The cells which express the encoded polypeptide will be fluorescently labeled and easily visualized under the microscope. See, for example, Hashido et al. (1992) Biochem. Biophys. Res. Comm. 187:1241-1248.

The present invention further provides methods for detecting the presence of and/or measuring a level of a polypeptide in a biological sample. The methods generally comprise: a) contacting the sample with an antibody specific for a differentially expressed polypeptide in a test cell; and b) detecting binding between the antibody and molecules of the sample. The level of antibody binding (either qualitative or quantitative) indicates the cancerous state of the cell. For example, where the differentially expressed gene is increased in cancerous cells, detection of an increased level of antibody binding to the test sample relative to antibody binding level associated with a normal cell indicates that the test cell is cancerous.

Suitable controls include a sample known not to contain the encoded polypeptide; and a sample contacted with an antibody not specific for the encoded polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay.

In general, the specific antibody will be detectably labeled, either directly or indirectly. Direct labels include radioisotopes; enzymes whose products are detectable (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like.

The antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls, and to appropriate standards.

In some embodiments, the methods are adapted for use in vivo, e.g., to locate or identify sites where cancer cells are present. In these embodiments, a detectably-labeled moiety, e.g., an antibody, which is specific for a cancer-associated polypeptide is administered to an individual (e.g., by injection), and labeled cells are located using standard imaging techniques, including, but not limited to, magnetic resonance imaging, computed tomography scanning, and the like. In this manner, cancer cells are differentially labeled.

In some embodiments, methods are provided for detecting a cancer cell by detecting expression in the cell of a transcript or that is differentially expressed in a cancer cell. Any of a variety of known methods can be used for detection, including, but not limited to, detection of a transcript by hybridization with a polynucleotide that hybridizes to a polynucleotide that is differentially expressed in a cancer cell; detection of a transcript by a polymerase chain reaction using specific oligonucleotide primers; in situ hybridization of a cell using as a probe a polynucleotide that hybridizes to a gene that is differentially expressed in a cancer cell and the like.

In many embodiments, the levels of a subject gene product are measured. By measured is meant qualitatively or quantitatively estimating the level of the gene product in a first biological sample either directly (e.g. by determining or estimating absolute levels of gene product) or relatively by comparing the levels to a second control biological sample. In many embodiments the second control biological sample is obtained from an individual not having not having cancer. As will be appreciated in the art, once a standard control level of gene expression is known, it can be used repeatedly as a standard for comparison. Other control samples include samples of cancerous tissue.

The methods can be used to detect and/or measure mRNA levels of a gene that is differentially expressed in a cancer cell. In some embodiments, the methods comprise: contacting a sample with a polynucleotide that corresponds to a differentially expressed gene described herein under conditions that allow hybridization; and detecting hybridization, if any. Detection of differential hybridization, when compared to a suitable control, is an indication of the presence in the sample of a polynucleotide that is differentially expressed in a cancer cell. Appropriate controls include, for example, a sample that is known not to contain a polynucleotide that is differentially expressed in a cancer cell. Conditions that allow hybridization are known in the art, and have been described in more detail above.

Detection can also be accomplished by any known method, including, but not limited to, in situ hybridization, PCR (polymerase chain reaction), RT-PCR (reverse transcription-PCR), and “Northern” or RNA blotting, arrays, microarrays, etc, or combinations of such techniques, using a suitably labeled polynucleotide. A variety of labels and labeling methods for polynucleotides are known in the art and can be used in the assay methods of the invention. Specific hybridization can be determined by comparison to appropriate controls.

Polynucleotides described herein are used for a variety of purposes, such as probes for detection of and/or measurement of, transcription levels of a polynucleotide that is differentially expressed in a cancer cell. A probe that hybridizes or amplifies specifically a polynucleotide disclosed herein should provide a detection signal at least 2-, 5-, 10-, or 20-fold higher than the background hybridization provided with other unrelated sequences. It should be noted that “probe” as used in this context of detection of nucleic acid is meant to refer to a polynucleotide sequence used to detect a differentially expressed gene product in a test sample. As will be readily appreciated by the ordinarily skilled artisan, the probe can be detectably labeled and contacted with, for example, an array comprising immobilized polynucleotides obtained from a test sample (e.g., mRNA). Alternatively, the probe can be immobilized on an array and the test sample detectably labeled. These and other variations of the methods of the invention are well within the skill in the art and are within the scope of the invention.

Labeled nucleic acid probes may be used to detect expression of a gene corresponding to the provided polynucleotide, e.g. in a macroarray format, Northern blot, etc. The amount of hybridization can be quantitated to determine relative amounts of expression, for example under a particular condition. Probes are used for in situ hybridization to cells to detect expression. Probes can also be used in vivo for diagnostic detection of hybridizing sequences. Probes may be labeled with a radioactive isotope. Other types of detectable labels can be used such as chromophores, fluorophores, and enzymes.

PCR is another means for detecting small amounts of target nucleic acids, methods for which may be found in Sambrook, et al. Molecular Cloning: A Laboratory Manual, CSH Press 1989, pp. 14.2-14.33. A detectable label may be included in the amplification reaction. The label may be conjugated to one or both of the primers. Alternatively, the pool of nucleotides used in the amplification is labeled, so as to incorporate the label into the amplification product.

Polynucleotide arrays provide a high throughput technique that can assay a large number of polynucleotides or polypeptides in a sample. This technology can be used as a tool to test for differential expression. A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions.

### Diagnosis, Prognosis, Assessment of Therapy (Therametrics), and Management of Cancer

The polynucleotides described herein, as well as their gene products and corresponding genes and gene products, are of particular interest as genetic or biochemical markers (e.g., in blood or tissues) that will detect the changes along the carcinogenesis pathway and/or to monitor the efficacy of various therapies and preventive interventions.

For example, the level of expression of certain polynucleotides can be indicative of a poorer prognosis, and therefore warrant more aggressive chemo- or radio-therapy for a patient or vice versa. The correlation of novel surrogate tumor specific features with response to treatment and outcome in patients can define prognostic indicators that allow the design of tailored therapy based on the molecular profile of the tumor. These therapies include antibody targeting, antagonists (e.g., small molecules), and gene therapy.

Determining expression of certain polynucleotides and comparison of a patient's profile with known expression in normal tissue and variants of the disease allows a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the genes corresponding to the polynucleotides described herein are staging of the cancerous disorder, and grading the nature of the cancerous tissue.

The polynucleotides that correspond to differentially expressed genes, as well as their encoded gene products, can be useful to monitor patients having or susceptible to cancer to detect potentially malignant events at a molecular level before they are detectable at a gross morphological level. In addition, the polynucleotides described herein, as well as the genes corresponding to such polynucleotides, can be useful as therametrics, e.g., to assess the effectiveness of therapy by using the polynucleotides or their encoded gene products, to assess, for example, tumor burden in the patient before, during, and after therapy.

Furthermore, a polynucleotide identified as corresponding to a gene that is differentially expressed in, and thus is important for, one type of cancer can also have implications for development or risk of development of other types of cancer, e.g., where a polynucleotide represents a gene differentially expressed across various cancer types. Thus, for example, expression of a polynucleotide corresponding to a gene that has clinical implications for SCCC might also have clinical implications for metastatic breast cancer, colon cancer, or ovarian cancer, etc.

Staging. Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

The polynucleotides and corresponding genes and gene products described herein can facilitate fine-tuning of the staging process by identifying markers for the aggressiveness of a cancer, e.g. the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

Grading of cancers. Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors generally being more aggressive than well-differentiated or low-grade tumors.

The polynucleotides, and their corresponding genes and gene products, can be especially valuable in determining the grade of the tumor, as they not only can aid in determining the differentiation status of the cells of a tumor, they can also identify factors other than differentiation that are valuable in determining the aggressiveness of a tumor, such as metastatic potential. Low grade means that the cancer cells look very like the normal cells. They are usually slowly growing and are less likely to spread. In high grade tumors the cells look very abnormal. They are likely to grow more quickly and are more likely to spread.

Assessment of proliferation of cells in tumor. The differential expression level of the polynucleotides described herein can facilitate assessment of the rate of proliferation of tumor cells, and thus provide an indicator of the aggressiveness of the rate of tumor growth. For example, assessment of the relative expression levels of genes involved in cell cycle can provide an indication of cellular proliferation, and thus serve as a marker of proliferation.

Detection of Cancer.

The polynucleotides corresponding to genes that exhibit the appropriate expression pattern can be used to detect cancer in a subject. The expression of appropriate polynucleotides can be used in the diagnosis, prognosis and management of cancer. Detection of cancer can be determined using expression levels of any of these sequences alone or in combination with the levels of expression of other known cancer genes. Determination of the aggressive nature and/or the metastatic potential of a cancer can be determined by comparing levels of one or more gene products of the genes corresponding to the polynucleotides described herein, and comparing total levels of another sequence known to vary in cancerous tissue. Expression of specific marker polynucleotides can be used to discriminate between normal and cancerous tissue, to discriminate between cancers with different cells of origin, to discriminate between cancers with different potential metastatic rates, etc. For a review of other markers of cancer, see, e.g., Hanahan et al. (2000) Cell 100:57-70.

Treatment of Cancer

The invention further provides methods for reducing growth of cancer cells. The methods provide for decreasing the expression of a gene that, is differentially expressed in a cancer cell or decreasing the level of and/or decreasing an activity of a cancer-associated polypeptide. In general, the methods comprise contacting a cancer cell with a substance that modulates expression of a gene that is differentially expressed in cancer; or a level of and/or an activity of a cancer-associated polypeptide.

“Reducing growth of cancer cells” includes, but is not limited to, reducing proliferation of cancer cells, and reducing the incidence of a non-cancerous cell becoming a cancerous cell. Whether a reduction in cancer cell growth has been achieved can be readily determined using any known assay, including, but not limited to, [³H]-thymidine incorporation; counting cell number over a period of time; detecting and/or measuring a marker associated with cervical cancer, etc.

The present invention provides methods for treating cancer, generally comprising administering to an individual in need thereof a substance that reduces cancer cell growth, in an amount sufficient to reduce cancer cell growth and treat the cancer. Whether a substance, or a specific amount of the substance, is effective in treating cancer can be assessed using any of a variety of known diagnostic assays for cancer, including, but not limited to, proctoscopy, rectal examination, biopsy, contrast radiographic studies, CAT scan, and detection of a tumor marker associated with cancer in the blood of the individual. The substance can be administered systemically or locally. Thus, in some embodiments, the substance is administered locally, and cancer growth is decreased at the site of administration. Local administration may be useful in treating, e.g., a solid tumor.

A substance that reduces cancer cell growth can be targeted to a cancer cell. Thus, in some embodiments, the invention provides a method of delivering a drug to a cancer cell, comprising administering a drug-antibody complex to a subject, wherein the antibody is specific for a cancer-associated polypeptide, and the drug is one that reduces cancer cell growth, a variety of which are known in the art. Targeting can be accomplished by coupling (e.g., linking, directly or via a linker molecule, either covalently or non-covalently, so as to form a drug-antibody complex) a drug to an antibody specific for a cancer-associated polypeptide. Methods of coupling a drug to an antibody are well known in the art and need not be elaborated upon herein.

Tumor Classification and Patient Stratification

The invention further provides for methods of classifying tumors, and thus grouping or “stratifying” patients, according to the expression profile of selected differentially expressed genes in a tumor. Differentially expressed genes can be analyzed for correlation with other differentially expressed genes in a single tumor type or across tumor types. Genes that demonstrate consistent correlation in expression profile in a given cancer cell type (e.g., in a cancer cell or type of cancer) can be grouped together, e.g., when one gene is overexpressed in a tumor, a second gene is also usually overexpressed. Tumors can then be classified according to the expression profile of one or more genes selected from one or more groups.

The tumor of each patient in a pool of potential patients can be classified as described above. Patients having similarly classified tumors can then be selected for participation in an investigative or clinical trial of a cancer therapeutic where a homogeneous population is desired. The tumor classification of a patient can also be used in assessing the efficacy of a cancer therapeutic in a heterogeneous patient population. In addition, therapy for a patient having a tumor of a given expression profile can then be selected accordingly.

The invention also encompasses the selection of a therapeutic regimen based upon the expression profile of differentially expressed genes in the patient's tumor. For example, a tumor can be analyzed for its expression profile of the genes described herein, e.g., the tumor is analyzed to determine which genes are expressed at elevated levels or at decreased levels relative to normal cells of the same tissue type. The expression patterns of the tumor are then compared to the expression patterns of tumors that respond to a selected therapy. Where the expression profiles of the test tumor cell and the expression profile of a tumor cell of known drug responsivity at least substantially match (e.g., selected sets of genes at elevated levels in the tumor of known drug responsivity and are also at elevated levels in the test tumor cell), then the therapeutic agent selected for therapy is the drug to which tumors with that expression pattern respond.

Pattern Matching in Diagnosis Using Arrays

In another embodiment, the diagnostic and/or prognostic methods of the invention involve detection of expression of a selected set of genes in a test sample to produce a test expression pattern. The test expression pattern is compared to a reference expression pattern, which is generated by detection of expression of the selected set of genes in a reference sample (e.g., a positive or negative control sample). The selected set of genes includes at least one of the genes of the invention, which genes correspond to the polynucleotide sequences described herein. Of particular interest is a selected set of genes that includes gene differentially expressed in the disease for which the test sample is to be screened.

The present invention also encompasses methods for identification of agents having the ability to modulate activity of a differentially expressed gene product, as well as methods for identifying a differentially expressed gene product as a therapeutic target for treatment of cancer.

Identification of compounds that modulate activity of a differentially expressed gene product can be accomplished using any of a variety of drug screening techniques. Such agents are candidates for development of cancer therapies. Of particular interest are screening assays for agents that have tolerable toxicity for normal, non-cancerous human cells. The screening assays of the invention are generally based upon the ability of the agent to modulate an activity of a differentially expressed gene product and/or to inhibit or suppress phenomenon associated with cancer (e.g., cell proliferation, colony formation, cell cycle arrest, metastasis, and the like).

Screening assays can be based upon any of a variety of techniques readily available and known to one of ordinary skill in the art. In general, the screening assays involve contacting a cancerous cell with a candidate agent, and assessing the effect upon biological activity of a differentially expressed gene product. The effect upon a biological activity can be detected by, for example, detection of expression of a gene product of a differentially expressed gene (e.g., a decrease in mRNA or polypeptide levels, would in turn cause a decrease in biological activity of the gene product). Alternatively or in addition, the effect of the candidate agent can be assessed by examining the effect of the candidate agent in a functional assay. For example, where the differentially expressed gene product is an enzyme, then the effect upon biological activity can be assessed by detecting a level of enzymatic activity associated with the differentially expressed gene product. The functional assay will be selected according to the differentially expressed gene product. In general, where the differentially expressed gene is increased in expression in a cancerous cell, agents of interest are those that decrease activity of the differentially expressed gene product.

Exemplary assays useful in screening candidate agents include, but are not limited to, hybridization-based assays (e.g., use of nucleic acid probes or primers to assess expression levels), antibody-based assays (e.g., to assess levels of polypeptide gene products), binding assays (e.g., to detect interaction of a candidate agent with a differentially expressed polypeptide, which assays may be competitive assays where a natural or synthetic ligand for the polypeptide is available), and the like. Additional exemplary assays include, but are not necessarily limited to, cell proliferation assays, antisense knockout assays, assays to detect inhibition of cell cycle, assays of induction of cell death/apoptosis, and the like. Generally such assays are conducted in vitro, but many assays can be adapted for in vivo analyses, e.g., in an animal model of the cancer.

The term “agent” as used herein describes any molecule, e.g. protein or pharmaceutical, with the capability of modulating a biological activity of a gene product of a differentially expressed gene. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting differentially expressed gene products) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Exemplary candidate agents of particular interest include, but are not limited to, antisense and RNAi polynucleotides, and antibodies, soluble receptors, and the like. Antibodies and soluble receptors are of particular interest as candidate agents where the target differentially expressed gene product is secreted or accessible at the cell-surface (e.g., receptors and other molecule stably-associated with the outer cell membrane).

For methods that involve RNAi (RNA interference), a double stranded RNA (dsRNA) molecule is usually used. The dsRNA is prepared to be substantially identical to at least a segment of a subject polynucleotide (e.g. a cDNA or gene). In general, the dsRNA is selected to have at least 70%, 75%, 80%, 85% or 90% sequence identity with the subject polynucleotide over at least a segment of the candidate gene. In other instances, the sequence identity is even higher, such as 95%, 97% or 99%, and in still other instances, there is 100% sequence identity with the subject polynucleotide over at least a segment of the subject polynucleotide. The size of the segment over which there is sequence identity can vary depending upon the size of the subject polynucleotide. In general, however, there is substantial sequence identity over at least 15, 20, 25, 30, 35, 40 or 50 nucleotides. In other instances, there is substantial sequence identity over at least 100, 200, 300, 400, 500 or 1000 nucleotides; in still other instances, there is substantial sequence identity over the entire length of the subject polynucleotide, i.e., the coding and non-coding region of the candidate gene.

Because only substantial sequence similarity between the subject polynucleotide and the dsRNA is necessary, sequence variations between these two species arising from genetic mutations, evolutionary divergence and polymorphisms can be tolerated. Moreover, as described further in, the dsRNA can include various modified or nucleotide analogs.

Usually the dsRNA consists of two separate complementary RNA strands. However, in some instances, the dsRNA may be formed by a single strand of RNA that is self-complementary, such that the strand loops back upon itself to form a hairpin loop. Regardless of form, RNA duplex formation can occur inside or outside of a cell.

The size of the dsRNA that is utilized varies according to the size of the subject polynucleotide whose expression is to be suppressed and is sufficiently long to be effective in reducing expression of the subject polynucleotide in a cell. Generally, the dsRNA is at least 10-15 nucleotides long. In certain applications, the dsRNA is less than 20, 21, 22, 23, 24 or 25 nucleotides in length. In other instances, the dsRNA is at least 50, 100, 150 or 200 nucleotides in length. The dsRNA can be longer still in certain other applications, such as at least 300, 400, 500 or 600 nucleotides. Typically, the dsRNA is not longer than 3000 nucleotides. The optimal size for any particular subject polynucleotide can be determined by one of ordinary skill in the art without undue experimentation by varying the size of the dsRNA in a systematic fashion and determining whether the size selected is effective in interfering with expression of the subject polynucleotide. dsRNA can be prepared according to any of a number of methods that are known in the art, including in vitro and in vivo methods, as well as by synthetic chemistry approaches.

Pharmaceutical compositions can comprise polypeptides, receptors that specifically bind a polypeptide produced by a differentially expressed gene (e.g., antibodies, or polynucleotides (including antisense nucleotides and ribozymes) of the claimed invention in a therapeutically effective amount. The compositions can be used to treat primary tumors as well as metastases of primary tumors. In addition, the pharmaceutical compositions can be used in conjunction with conventional methods of cancer treatment, e.g., to sensitize tumors to radiation or conventional chemotherapy.

Where the pharmaceutical composition comprises a receptor (such as an antibody) that specifically binds to a gene product encoded by a differentially expressed gene, the receptor can be coupled to a drug for delivery to a treatment site or coupled to a detectable label to facilitate imaging of a site comprising cancer cells. Methods for coupling antibodies to drugs and detectable labels are well known in the art, as are methods for imaging using detectable labels.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature.

The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic composition agents includes local or systemic administration, including injection, oral administration, particle gun or catheterized administration, and topical administration.

Also provided by the subject invention are kits for practicing diagnostic and therapeutic methods. The subject kits include at least one or more of: a subject nucleic acid, isolated polypeptide or an antibody thereto. Other optional components of the kit include: restriction enzymes, control primers and plasmids; buffers, cells, carriers, adjuvants etc. The nucleic acids of the kit may also have restriction sites, multiple cloning sites, primer sites, etc to facilitate their ligation into other plasmids. The various components of the kit may be present in separate containers or certain compatible components may be precombined into a single container, as desired. In certain embodiments, controls, such as samples from a cancerous or non-cancerous cell are provided by the invention. Further embodiments of the kit include an antibody for a subject polypeptide and a chemotherapeutic agent to be used in combination with the polypeptide as a treatment.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### A Panel of Genes Transcriptionally Upregulated in Squamous Cell Carcinoma of the Cervix Identified by RDA, Confirmed by Macroarray, and Validated by Real-Time Quantitative RT-PCR

The research presented here uses representational difference analysis (RDA) to isolate a relatively small candidate pool of transcripts upregulated in disease versus normal tissue in a single patient. RDA has been used to identify potentially upregulated transcripts in other cancers. The selected pool of candidate transcripts is then screened by comparative hybridization on DNA macroarrays with amplified cDNA from the original patient from which they were derived and seven other patients. Real-time quantitative RT-PCR is firmly established as a highly sensitive gene-specific method for determining transcript levels of selected genes and is used here to confirm the transcriptional upregulation of several of the genes identified by the RDA procedure across multiple patients.

Methods

Patient specimens: Tissue specimens were obtained from ILSBio (Chestertown, Md.) or Genomics Collaborative (Cambridge, Mass.). All patient samples were obtained in Vietnam and collected with patient consent in compliance with the company IRBs and with the Code of Federal Regulations (CFR) 45CFR46.101B. All specimens were anonymized by ILS Bio and Genomics Collaborative. Paired SCCC (disease) and non-cancer (normal) tissues were taken from single patient surgical specimens that had been frozen in liquid nitrogen within 30 minutes of extirpation. Microscope slides were reviewed by a pathologist for diagnosis and staging, and a pathology report was received with each tissue specimen.

RNA isolation: Frozen tissue samples (150 mg) were ground to a fine powder under liquid nitrogen. Total RNA was isolated from the powder using an RNEasy Midi Kit (Qiagen, Valencia, Calif.). Samples were treated with DNAse during the column purification procedure. Total RNA samples were analyzed using an Agilent (Palo Alto, Calif.) 2100 Bioanalyzer system for 18S and 28S band integrity, quantitated by A₂₈₀ absorbance, and checked for purity by A₂₆₀/A₂₈₀ ratio.

Poly-A RNA isolation: Messenger RNA was isolated from total RNA of three patients (A00330, VNM105, and VNM269) using Boehringer-Mannheim's (Gaithersburg, Md.) magnetic bead isolation kit essentially according to manufacturer's instructions. The bound mRNA was washed extensively with high salt buffer and eluted with water. The purity and quantity of mRNA were estimated by A₂₆₀/A₂₈₀ nm readings.

cDNA synthesis: cDNA was synthesized according to one of two methods. The first method essentially followed that outlined in (Gubler (1987) Methods Enzymol. 152:325-9) using 2 μM dT18-NOT-B primer (5′ biotin-CACACACACACACAGGGCCGCT₍₁₈₎-3′) with poly-A mRNA from normal and disease tissues from patients A00330, VNM105, and VNM269. In the second method, 5 μg of total RNA from normal and disease tissues of patients VNM105, VNM269, VNM095, VNM098, VNM277, VNM279, and VNM285 was used as template in the Roche (Indianapolis, Iowa) cDNA Synthesis System according to manufacturer's instructions using the d_{T18}-NOT-B primer.

RDA subtraction: RDA protocols were carried out as described by Hubank using cDNA from normal and disease tissues of patient A00330. Normal and disease amplicons were subsequently used to generate melt depletion normal and melt depletion disease amplicons. Subtraction-hybridization reactions were performed using reduced amounts of amplicon. Hybridization reactions used normal, disease, melt depletion normal, or melt depletion disease amplicon as driver. Two rounds of subtraction were performed using tester:driver ratios of 1:80 and 1:400. RDA products from the second round of hybridization from reactions using normal or melt depletion normal driver conditions were shotgun-cloned into the BamHI site of pBluescript II KS+. Three groups of 96 clones were selected for analysis.

Plasmid purification: Bacterial colonies were picked and grown overnight in 2 ml LB supplemented with 100 ng/ml ampicillin at 37° C. with shaking. Plasmid DNA was purified with Qiagen plasmid miniprep columns according to manufacturer's instructions. Sequencing: Purified clones were sequenced using Applied Biosystems (Foster City, Calif.) Big Dye PCR reactions with either T3 22-mer (5′-GAAATTAACCCTCACTAAAGGG-3′) or T7 22-mer (5′-GTAATACGACTCACTATAGGGC-3′). The sequencing products were analyzed on ABI Prism 373 or 377 sequencers (Applied Biosytems).

PCR amplification of clones: Plasmids were confirmed by PCR amplification of the T3-T7 region of pBluescript II KS+ with T3 22-mer and T7 22-mer primers. Plasmids were amplified for 37 cycles of 95° C. for 10 seconds, 55° C. for 10 seconds, 72° C. for 50 seconds. Amplified fragments were confirmed for size and concentration by electrophoresis in 2% agarose gels or an Agilent 2100 Bioanalyzer system.

Amplicon probe synthesis: Ten to fifty percent of each cDNA reaction (normal and disease from all patients) was digested with DpnII and ligated to an excess of R-Bgl-12/24 linker. The resulting linkered cDNA was amplified essentially as described in the RDA amplicon generation protocol with the following modifications. All amplifications contained 5 units of Taq polymerase/100 μl reaction and 100 pM R-Bgl-24 primer. The number of cycles of amplification was determined based upon the amount of cDNA used as template (18 cycles for 1 μl of 6 μg/ml target). The cDNA concentrations were estimated based upon the amount of total RNA used for cDNA synthesis, using values of 2% poly-A mRNA and 100% cDNA synthesis efficiency. Eight reactions each were performed with normal and disease cDNA from each patient as template. Normal and disease amplicons were separately pooled, phenol/chloroform extracted, ethanol precipitated, resuspended and quantitated by _{A260} and checked for purity by A₂₆₀/A₂₈₀ ratio.

Purified amplicons were biotin-labeled to high specificity using Invitrogen's BioPrime labeling kit. Manufacturer's instructions were followed with the following exceptions: one microgram of template was used, and the label reactions were incubated for 90 to 120 minutes. The biotinylated product was purified away from free dNTPs and primers with BDBioscience/Clontech's (Palo Alto, Calif.) Chromaspin TE-100 size exclusion columns pre-equilibrated with 2×SSC/0.1% SDS. The average yield for probe synthesis was 10-12 μg/r×n as determined by biotin quantitation using KPL's (Gaithersburg, Md.) probe biotinylation kit.

DNA macroarray synthesis and hybridization: Paired DNA macroarrays with identical spot patterns were prepared with a Bio-Blot apparatus (BioRad, Hercules, Calif.) according to manufacturer's instructions. 1600 ng of each DNA sample including the MCS region of pBluescript II KS+was denatured in a solution of 0.4M NaOH in 2×SSC in a total volume of 440 μl and 110 μl was applied to paired spots on duplicate positively charged nylon membranes (Sigma-Aldrich, Dorset, UK). For all of the 65 gene fragments to be analyzed by macroarray in duplicate, it was necessary to use two separate pairs of membranes. Macroarrays were cross-linked in a UV Stratalinker (Stratagene, La Jolla, Calif.) at a setting of 1200×100 μJ.

Macroarray hybridization experiments were normalized by adding equal masses of normal and disease probe as determined by biotin quantitation to the hybridization reactions. Macroarrays were prehybridized in a roller bottle oven at 50° C. in 8 ml of 33% formamide in 2×SSC plus 200 ng/ml sheared salmon sperm DNA and 1.25 μg/ml DpnII-digested PCR product of the pBluescript II KS+MCS for at least an hour. 1 to 4 μg of normal or disease probe was denatured and added. Hybridizations were performed at 50° C. for >40 hours. The macroarrays were subjected to stringent wash conditions (three thirty-minute washes of 2×SSC 0.1% SDS at 50° C., one thirty-minute wash in 0.2×SSC 0.1% SDS at 45° C., and one hour-long wash in 2×SSC at room temperature) and developed with KPL's DNA Detector HRPO kit essentially according to manufacturer's instructions. Wash times were increased to 10 to 15 minutes, and the KPL chemiluminescent substrate was replaced with Pierce (Rockford, Ill.) SuperSignal West Dura Extended substrate. Luminescence was captured with Kodak (Rochester, N.Y.) Bio-Max film. Exposure times ranged from one second to twenty minutes.

Semiquantitative DNA macroarray analysis: Films were scanned as 300 dots per inch TIFF files using a Perfection 1250 flatbed printer (Epson America, Long Beach, Calif.). Images were analyzed for integrated optical density (intensity) in GelPro 3.1 (Media Cybernetics, North Reading, Mass.) using the dot blot analysis tools. Dot diameter was set at 90 and background close to the dot was subtracted. Average intensities for pairs of dots were recorded for normal and disease for each exposure. Semiquantitative fold expression values were calculated for each gene by dividing average disease intensity by average normal intensity at each exposure. Final values were chosen as those farthest from one, obtained preferably from exposures at which both normal and disease intensities were above background.

Real-time quantitative RT-PCR: Eleven genes were selected for real-time quantitative RT-PCR analysis using ABI's TaqMan system. Gene fragments were selected as candidates for analysis if DNA macroarray analysis indicated transcriptional upregulation in at least four of the eight patients. External primers and dual-labeled FAM-TAMRA internal probes were designed using ABI's Primer Express software based upon the sequence of the gene fragments isolated by the RDA procedure. Sequences of the primers and probes used are listed in Table 1. Template consisted of a 1/10 dilution of double-stranded cDNA into tRNA buffer (10 mM Tris pH 8.0, 5 μg/ml purified yeast tRNA). All patient cDNA samples (normal and disease) were normalized based on equal target input of total RNA into cDNA reactions (5 μg). Patient A00330 normal and disease amplicons were normalized by concentration calculated by A₂₆₀ absorption, before dilution to 0.2 ng/ul with tRNA buffer. Individual amplifications were performed in duplicate 30 μl reactions containing 90 nM external primers, 25 nM reporter probe, and 1.5 μl of template. Gene-specific quantitative calibration standards consisted of purified PCR products of the individual gene fragments isolated in the RDA protocols. PCR products were purified and diluted in tRNA buffer to establish a dilution series of 2×10⁷ copies/μl, 2×10⁶ copies/μl, and 2×10⁵ copies/μl for each gene fragment assay. Standards were tested for uniform differences between _{CT} values of 1/10 dilutions prior to use as quantitative standards.

TABLE 1

| | | | Internal FAM- |
|---|---|---|---|
| Gene | 5′ primer | 3′ primer | TAMRA probe |
| KRT16 | GGTCACGCATCTCATTCAGGA | ACGTGGAGATGGATGCTGC | CGGCTCAGGTCCACGCCAGG |
| ZWINT | AGGCCCAAGGCOAGTACC | AGGAGCTGGGACTGGTTTGA | CCTCCCCATCTGCACACCCTGTG |
| KRT14 | ATGTGCACGATGGCAAGGT | GGCAGCCTCAGTTCTTGGTG | TGTCCACCCACGAGCAGGTCCTTC |
| KARS | AACAGCTAAGCCAAGCCACTG | TCCTCAGGACCCACACCATT | TGCTGCCACCAACCACACCACTG |
| SPINT2 | TTTCTTTCCTCCAGGTAGAGTTTTCT | AACGATTCCAACATCACTTCTGTG | |
| HPV16E7 | | | |
| CDCA8 | CCAGAGGCCTTGGGAAACAT | TCCGTATGCTGCTGCAGATT | AAGAAGCTCTCCAACCGTCTCGCCC |
| G1P2 | CCTGCTGGTGGTGGACAAAT | GCTGCGGCCCTTGTTATTC | CGACGAACCTCTGAGCATCCTGGTGA |
| CCNB1 | GCAAGCAGTCAGACCAAAATACCT | CTAGCCAGTCAATTAGGATGGCTC | CTGGGTCGGGAAGTCACTGGAAACATG |
| AURKB | TGCTGACTTCGGCTGGTCT | AGGGTGCCACACATTGTCTTC | TGCATGCGCCCTCCCTGAGG |
| OAZ1 | ATAGCCACTGCTTCGCCAGA | CTGTGCAGGCTTAGGAGCG | AACCCAGCGCCACCATCCACG |
| ACTIN | GGGAAATCGTGCGTGACATT | TGGCCATCTCTTGCTCGAAG | AGGAGAAGCTGTGCTACGTCGCCCTG |

Assays were performed such that each plate tested two separate genes for normal and disease samples in each of eight patients, and included both gene-specific quantitative standards. Reactions were run on an ABI 7700 thermocycler. Each gene fragment was analyzed at least twice.

Calculated copy numbers based on each gene-specific quantitative standard were exported into Microsoft Excel (Microsoft, Seattle, Wash.) for statistical analysis. Copy numbers for each patient sample (normal and disease) were averaged and analyzed. Values that exceeded four times the standard deviation for any sample were removed. The remaining data always consisted of at least three values per sample. New averages and standard deviations were calculated for each sample, and each sample dataset was confirmed to have a coefficient of variation below 40%. Ratios of transcriptional upregulation (disease/normal) were calculated from the average copy numbers for each patient for each gene.

Validation of relative expression ratios in amplicon compared to cDNA: Double-stranded cDNA was synthesized from normal and disease samples from patient VNM285 using 6 μg total RNA and a novel poly-T18-based primer in a Roche cDNA synthesis system. Amplicons were generated as described above using 10% of the synthesized cDNA in a 22-cycle amplification reaction. Real-time quantitative RT-PCR was performed using normal and disease amplicons and cDNA as templates. The gene fragments for CCNB1, SPINT2, ZWINT, and ACTIN were amplified using the primers and probes listed in Table 1 as described above with the following differences. Normal and disease cDNAs were diluted 1/30 for use as template. Reactions were performed in triplicate in each assay. Each test gene was assayed in parallel with actin. Gene-specific ladders consisted of plasmid containing the fragment of interest in the dilution series described above. Two assays were performed for each gene.

Copy numbers were exported to an Excel spreadsheet and analyzed as described above. Amplicon copies for the test genes were adjusted based upon measured actin leveles in cDNA compared to amplicon. Corrected disease/normal ratios derived from cDNA and amplicon templates for each gene fragment were averaged, and coefficients of variation between the ratios for the cDNA and amplicon templates were calculated for each gene fragment.

Results

Representational difference analysis (RDA). RDA was performed using disease and normal tissues from a single patient (A00330) diagnosed with non-keratinizing SCCC. 288 clones were picked and were found to contain fragments matching portions of sixty-five different genes. Sixty-two of these are human genes, four of which are novel transcripts. The isolated gene fragments are listed in Table 2.

TABLE 2
TABLE 2. Summary report: DNA macroarray results of 65 genes analyzed by DNA macroarray.
The isolated fragments match the listed portions of the genes with at least 95% identity.
Position: the portion of the gene (indicated by nucleotide numbers) spanned by the RDA
fragment. Group I: genes upregulated in at least half the patients. Group II: genes upregulated
in less than half the patients.
Group I

| | | | VNM | VNM | VNM | VNM | VNM | A00 | VNM | VNM | Patients |
|---|---|---|---|---|---|---|---|---|---|---|---|
| Gene name | Locus | Position | 095 | 098 | 279 | 277 | 285 | 330* | 105 | 269 | upregulated |
| CCNB1 | NM_031966 | 664-865 | + | +++ | ++ | +++ | +++ | +++ | + | ++ | 8 |
| KRT14 | NM_000526 | 1362-1531 | +++ | +++ | + | +++ | = | +++ | + | ++ | 7 |
| ALDH3A1 | NM_000691 | 1044-1277 | +++ | −−− | + | = | + | +++ | +++ | ++ | 6 |
| CALML5 | NM_017422 | 475-822 | +++ | = | − | + | ++ | +++ | ++ | +++ | 6 |
| EIF4A1 | NM_001416 | 305-604 | ++ | + | = | +++ | + | − | ++ | + | 6 |
| HNRPM1 | NM_005968 | 1529-1878 | + | + | = | ++ | + | = | + | + | 6 |
| KARS | NM_005548 | 103-398 | +++ | = | − | + | +++ | +++ | + | +++ | 6 |
| KRT16 | NM_005557 | 799-1204 | +++ | +++ | +++ | +++ | +++ | ++ | = | = | 6 |
| NDRG1 | NM_006096 | 992-1330 | − | +++ | ++ | +++ | +++ | + | +++ | | |
| OAZ1 | NM_004152 | 84-292 | = | +++ | = | +++ | ++ | + | ++ | ++ | 6 |
| SPINT2 | NM_021102 | 960-1149 | = | +++ | − | +++ | +++ | + | + | +++ | 6 |
| TKT | NM_001064 | 306-744 | +++ | +++ | ++ | ++ | = | +++ | +++ | | |
| ZNF9 | NM_003418 | 134-307 | +++ | ++ | − | +++ | −−− | + | ++ | +++ | 6 |
| ZWINT | NM_032997 | 1209-1450 | +++ | +++ | + | +++ | = | ++ | +++ | | |
| AP2M1 | NM_004068 | 420-588 | 0 | + | −− | + | 0 | + | + | + | 5 |
| CBR1 | NM_001757 | 753-1035 | +++ | * | − | + | −−− | +++ | ++ | ++ | 5 |
| CES1 | NM_001266 | 1539-1777 | +++ | = | − | + | −−− | +++ | + | ++ | 5 |
| FDX1 | NM_004109 | 231-497 | + | +++ | + | + | − | = | − | ++ | 5 |
| G1P2 | NM_005101 | 186-382 | 0 | +++ | +++ | +++ | −− | +++ | ++ | −−− | 5 |
| GAPDH | NM_002046 | 316-513 | +++ | +++ | = | +++ | − | = | +++ | +++ | 5 |
| KRT13 | NM_153490 | 182-381 | +++ | | | | | | | | |
| KRT6A | NM_005554 | 930-1200 | +++ | +++ | ++ | +++ | | | | | |
| NQO1 | NM_000903 | 530-860 | −−− | + | = | + | = | +++ | +++ | | |
| P4HB | NM_000918 | 1068-1494 | +++ | + | = | +++ | | | | | |
| PGDH | NM_002631 | 765-1002 | +++ | − | − | +++ | −−− | +++ | + | +++ | 5 |
| S100A9 | NM_002965 | 175-409 | +++ | − | −− | + | = | + | + | +++ | 5 |
| TALDO1 | NM_006755 | 779-986 | ++ | − | − | + | = | +++ | + | +++ | 5 |
| 18S rRNA | XO3205 | 312-592 | +++ | +++ | +++ | = | −− | * | − | +++ | 4 |
| AURKB | NM_004217 | 594-823 | 0 | 0 | 0 | +++ | | | | | |
| CDCA8 | NM_018101 | | | | | | | | | | |
| cDNA DKFZp686O2421 | BX648076 | 431-716 | +++ | | | | | | | | |
| FLJ23841 | NM_144589 | 502-823 | +++ | = | = | ++ | = | + | = | +++ | 4 |
| HM74 | NM_006018 | 288-477 | + | 0 | 0 | ++ | +++ | * | 0 | +++ | 4 |
| HPV16E7 | AF003020 | 63-315 | 0 | +++ | 0 | 0 | +++ | +++ | | | |
| MGC14799 | NM_032336 | 441-688 | | | | | | | | | |
| MYBL2 | NM_002466 | | | | | | | | | | |
| PSMD4 | NM_002810 | 34-314 | 0 | ++ | = | ++ | 0 | = | + | ++ | 4 |
| SPATA11 | NM_032306 | 578-925 | + | 0 | = | + | − | ++ | = | +++ | 4 |
| TNFS10 | NM_003810 | 1483-1643 | +++ | | | | | | | | |
| TUBG1 | NM_001070 | | | | | | | | | | |
| Yif1p | NM_033557 | 904-1206 | − | 0 | 0 | +++ | | | | | |
| AKR1B10 | NM_020299 | 1012-1316 | +++ | −− | = | + | = | +++ | * | = | 3 |
| ARHGAP4 | NM_001666 | 1701-2119 | 0 | ++ | 0 | = | + | 0 | = | ++ | 3 |
| ASF1B | NM_018154 | 1075-1261 | 0 | 0 | 0 | +++ | = | = | + | + | 3 |
| DTYMK | NM_012145 | 788-974 | 0 | 0 | 0 | ++ | = | + | = | + | 3 |
| FLJ10156 | NM_019013 | 93-441 | 0 | 0 | 0 | − | − | + | + | ++ | 3 |
| H17 | NM_017547 | 618-845 | 0 | 0 | 0 | + | − | = | + | +++ | 3 |
| JFC1 | NM_032872 | 782-995 | 0 | 0 | 0 | ++ | 0 | = | + | +++ | 3 |
| MCG10911 | NM_032302 | 578-776 | + | 0 | 0 | + | = | − | 0 | + | 3 |
| MCM2 3′ | NM_004526 | 912-1201 | 0 | 0 | 0 | + | 0 | +++ | + | * | 3 |
| novel transcript | AY714068 | 1-276 | 0 | 0 | 0 | 0 | 0 | + | 0 | + | 2 |
| ACO2 | NM_001098 | 2099-2424 | 0 | 0 | 0 | ++ | 0 | −− | = | ++ | 2 |
| cDNA DKFZp434B0425 | AL157459 | 1252-1508 | 0 | 0 | 0 | +++ | + | − | 0 | 0 | 2 |
| NEFL | NM_006158 | 1053-1268 | 0 | 0 | 0 | 0 | 0 | ++ | 0 | + | 2 |
| NOD9 | NM_024618 | 2451-2978 | + | = | = | = | − | +++ | = | * | 2 |
| PP3856 | NM_145201 | 270-474 | 0 | 0 | 0 | = | 0 | − | + | + | 2 |
| RAPGEFL1 | NM_016339 | 765-1014 | ++ | = | −− | = | −−− | +++ | = | * | 2 |
| novel transcript | AY714069 | 1-180 | 0 | 0 | 0 | 0 | 0 | −−− | −− | ++ | 1 |
| E. coli K12 BHB2600 | ECOHU47 | 50461-50662 | 0 | 0 | 0 | 0 | 0 | − | 0 | + | 1 |
| novel transcript | AY714070 | 1-56 | 0 | 0 | 0 | 0 | 0 | −− | 0 | + | 1 |
| FLJ36635 | AK093954 | 799-1061 | 0 | 0 | 0 | − | = | * | = | ++ | 1 |
| RH BDF1 | NM_022450 | 1490-1764 | 0 | 0 | 0 | ++ | − | 0 | 0 | − | 1 |
| novel transcript | AY714071 | 1-152 | 0 | 0 | 0 | 0 | 0 | −− | 0 | = | 0 |
| OKL38 | NM_182981 | 781-10980 | 0 | 0 | 0 | 0 | 0 | = | 0 | 0 | 0 |
| S. aureus sabac-114 | AC025949 | 4276-4375 | 0 | 0 | 0 | 0 | 0 | 0 | 0 | = | 0 |

^{a}original RDA source.
+: Disease/normal intensity between 2 and 5 where normal intensity is above background; or normal intensity is at background level, disease intensity is above background, and disease intensity is up to 5-fold greater than measured normal background level.
++: Disease/normal intensity between 5.1 and 10.
+++: Disease/normal intensity above 10.
=: Disease/normal intensity between 0.55 and 1.7.
−: Normal/disease intensity between 2 and 5 where disease intensity is above background; or disease intensity is at background level, normal intensity is above background, and normal intensity is up to 5-fold greater than measured disease background level.
−−: Normal/disease intensity between 5.1 and 10.
−−−: Normal/disease intensity above 10.
0: Normal and disease intensities not detected above background.
*No conclusion (conflicting results across multiple hybridizations or clones).

Gene name in bold indicates that it was chosen for further study by real-time quantitative RT-PCR analysis. Genes are listed in order from those upregulated in the greatest number of patients to least. Patients are listed in order from those with the greatest number of upregulated genes to least.

Real-time quantitative RT-PCR validation of relative expression ratios in amplicon. An experiment was performed to determine whether the amplicons resulting from approximately 20,000-fold amplification of cDNA (e.g. 50 μg amplicon synthesized from 2.5 ng cDNA) maintained the same relative ratios of disease/normal expression as in the original cDNA. Three gene fragments (CCNB1, ZWINT, and SPINT2) were each tested in parallel with actin by real-time quantitative RT-PCR using normal and disease cDNA and normal and disease amplicons from a single patient (VNM285) as templates. The average coefficient of variation for calculated copy number was 7.09% (range 3.61% to 14.13%). Disease/normal ratios for the test genes were actin-corrected and compared with respect to template. As shown in Table 3, the disease/normal ratios for each gene are quite similar between amplicon and cDNA, with an average coefficient of variation of 15.8% (range 7.8% to 22.0%).

TABLE 3
TABLE 3. Retention of expression ratios in amplified cDNA.
Actin-corrected expression levels (disease/normal) of three gene
fragments were determined by real-time quantitative RT-PCR using
cDNA and amplicon as templates. Average expression levels between
the two templates and the coefficients of variation are listed.
Disease/normal ratio

| Gene name | Amplicon | cDNA | Coefficient of variation |
|---|---|---|---|
| CCNB1 | 4.63 | 3.38 | 22.0% |
| SPINT2 | 2.65 | 2.96 | 7.8% |
| ZWINT | 2.42 | 3.12 | 17.6% |

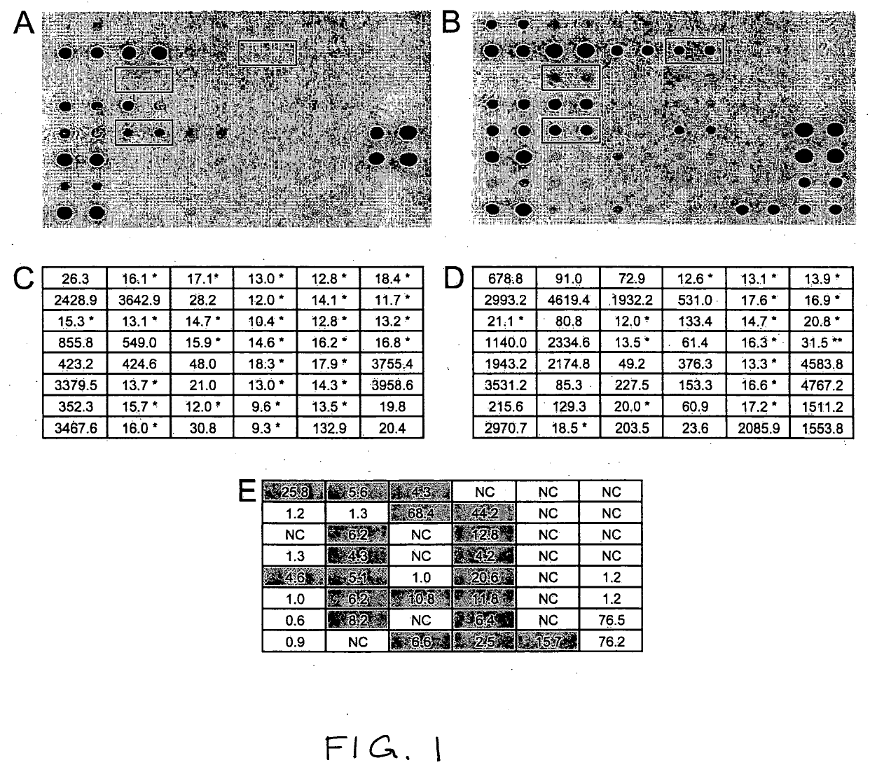

DNA macroarray analysis. Normal and disease biotinylated amplicon probes from patient A00330 and seven additional cervical cancer patients were hybridized to arrays of PCR products representing the RDA fragments. An example of one visualized and analyzed macroarray is shown in FIG. 1. Several pairs of dots are significantly darker in panel B (hybridized to disease amplicon) than the corresponding pairs in panel A (hybridized to normal amplicon). Additionally, several pairs of dots are easily visible in panel B that are not visible in panel A. Corresponding average intensity values are shown in panels C and D. The calculated fold change in expression (disease/normal) is shown in panel E. As is seen by the multiple cells highlighted in gray in panel E, the patient listed in FIG. 1 (VNM105) shows strong transcriptional upregulation in disease versus normal tissue at this exposure for many of the genes present on the blot. It should be noted that FIG. 1 displays a single exposure and provides an example of how the analysis is performed; whereas the data presented in Table 2 reflect data from multiple exposure times, generally ranging from one second to twenty minutes. The range of exposure times was required to capture the wide range of transcript levels seen among the genes isolated by the RDA procedure.

The results of the DNA macroarray analysis of the sixty-five gene fragments in the eight patients examined are shown in Table 2. Forty-one of the sixty-five genes isolated by RDA in the original patient (63.1%) are transcriptionally upregulated in at least half of all the patients Group I). Of these, fourteen genes (21.4%) are transcriptionally upregulated in at least seventy-five percent of the patients. The remaining 24 genes were transcriptionally upregulated in less than half the patients as determined by DNA macroarray analysis (Group II). It should be noted that many of the gene fragments listed in Group II were not detected in all patients by DNA macroarray analysis. Such a lack of detection does not necessarily indicate that the gene fragments are not transcriptionally upregulated in those patients, merely that the transcript levels were too low to be detected by this method.

Real-time quantitative RT-PCR analysis of selected genes. To validate the expression array, eleven of the genes that were indicated as transcriptionally upregulated in at least half the patients by DNA macroarray analysis were analyzed by real-time quantitative RT-PCR. The genes chosen are indicated in bold typeface in Table 2. Table 4 summarizes the results of the real-time quantitative RT-PCR analysis. All of the genes shown in Table 4 are transcriptionally upregulated by 1.8-fold or greater in at least four of the eight patients in disease versus normal tissue. Ten of those eleven genes are transcriptionally upregulated by 1.8-fold or greater in at least six of the eight patients. For two patients, VNM095 and VNM279, real-time quantitative RT-PCR analysis showed transcriptional upregulation in half or fewer of the genes examined, whereas all other patients show transcriptional upregulation in at least two-thirds of the genes examined. The coefficient of variation for the replicate copy numbers for these genes ranged from 2.3% to 38%, with an average value of 17.3%.

TABLE 4
Patient Identifier

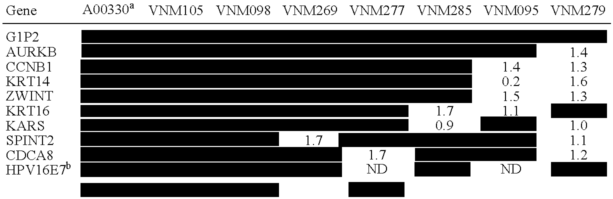

Real-time quantitative PCR analysis of ten selected genes Values indicate average fold transcriptional upregulation in disease versus normal for each gene in each patient as determined by dividing calculated average disease copy number by average normal copy number. Values of 1.8 or greater (indicating transcriptional upregulation) are highlighted by a gray background. Genes are listed by the fraction of patients in which transcriptional upregulation was detected.
^{a}original RDA source; amplicon used as template rather than cDNA.
^{b}HPV16E7 was detected in disease tissue but not normal tissue for five of the six patients, and so statistical analysis could not be applied.
unique: transcript detected in disease sample but not normal sample of that patient.
ND: transcript not detected in normal or disease sample of that patient.
Genes are listed in order from those upregulated in the greatest number of patients to least.
Patients are listed in order from those with the greatest number of upregulated genes to least.

This study was directed to investigate the phenotype of squamous cell carcinoma of the cervix (SCCC) by examining differences in expression between normal (non-cancerous) and disease (cancerous) cervical tissue. Pursuant to this goal, a panel of genes that are transcriptionally upregulated in SCCC was identified. A candidate group of sixty-five genes was identified by RDA using normal and disease tissues from a single patient. Amplicon probes were generated from normal and disease tissues in seven additional patients with SCCC and were used to confirm the transcriptional upregulation of this diverse gene set. The small amount of cDNA needed to generate the amplicon probe (<10 ng) for each patient sample allowed the remaining cDNA to be used in confirmatory real-time quantitative RT-PCR experiments. Forty-one of the sixty-five genes identified by RDA are transcriptionally upregulated in at least four of the eight patients as determined by comparative DNA macroarray hybridization analysis. Of the eleven genes examined by real-time quantitative RT-PCR, ten were confirmed to be transcriptionally upregulated in 75% of the patients, and one gene, OAZ1, was confirmed to be transcriptionally upregulated in 50% of the patients. The genes identified in this report are useful in diagnostic applications.

RDA subtraction using normal and disease tissues from a single patient reduced the transcriptome complexity and allowed the isolation of key candidates with the screening of relatively few clones (288). Other studies using RDA to isolate genes of interest have used pooled samples from several patients or used tissue culture samples. DNA macroarray analysis of the gene fragments isolated in the RDA protocols showed that more than two-thirds of these gene fragments appear to be transcriptionally upregulated in at least 50% of patients.

This result demonstrates the power of RDA to isolate a small number of genes of interest. This power is further demonstrated by the identification of four transcripts that were hitherto unknown and an additional four that are not represented on commercially available human arrays. The transcript levels of most of the genes in this group were too low to be detected by DNA macroarray. Determination of relative transcript levels of these genes could be examined by the sensitive methodology of real-time quantitative RT-PCR analysis.

Normal and disease amplicons from patient VNM285 that were used to generate biotinylated probe for hybridization experiments were directly compared with the original normal and disease cDNA by real-time quantitative RT-PCR. The results showed that amplicons have similar fold expression ratios (disease/normal) as compared to the cDNAs. The average coefficient of variation between the ratios was 15.8%, which is very small considering the high degree of amplification (approximately 20,000-fold) and the large increase in testable material.

The validated amplicons may be used in array hybridization and other expression analysis and diagnostic platforms, particularly in cases where the original source material is limiting.

Comparative hybridization of DNA macroarrays is identical in concept to comparative microarray hybridization, and carries similar potentials and dangers. Macroarrays have a limited number of spots available on each blot and thus limit the number of replicates possible for each gene. The macroarrays in this study consisted of relatively long DNA sequences (120 bp or more), and so present opportunities for cross-hybridization. cDNA-based microarrays share this quality but oligonucleotide-based microarrays do not. Macroarrays have some advantages over commercial microarrays. Macroarrays are inexpensive, straightforward to synthesize and use in a small laboratory, and can be stripped and reused several times. Macroarrays also allow the selective screening of a small number of genes, such as those isolated by RDA.

Eleven of the 45 genes were analysed by real time quantitative RT-PCR and confirmed to be upregulated in the cancerous specimen. These confirmatory results show that DNA macroarrays can be used in conjunction with RDA as a screening tool for identifying genes that are transcriptionally upregulated.

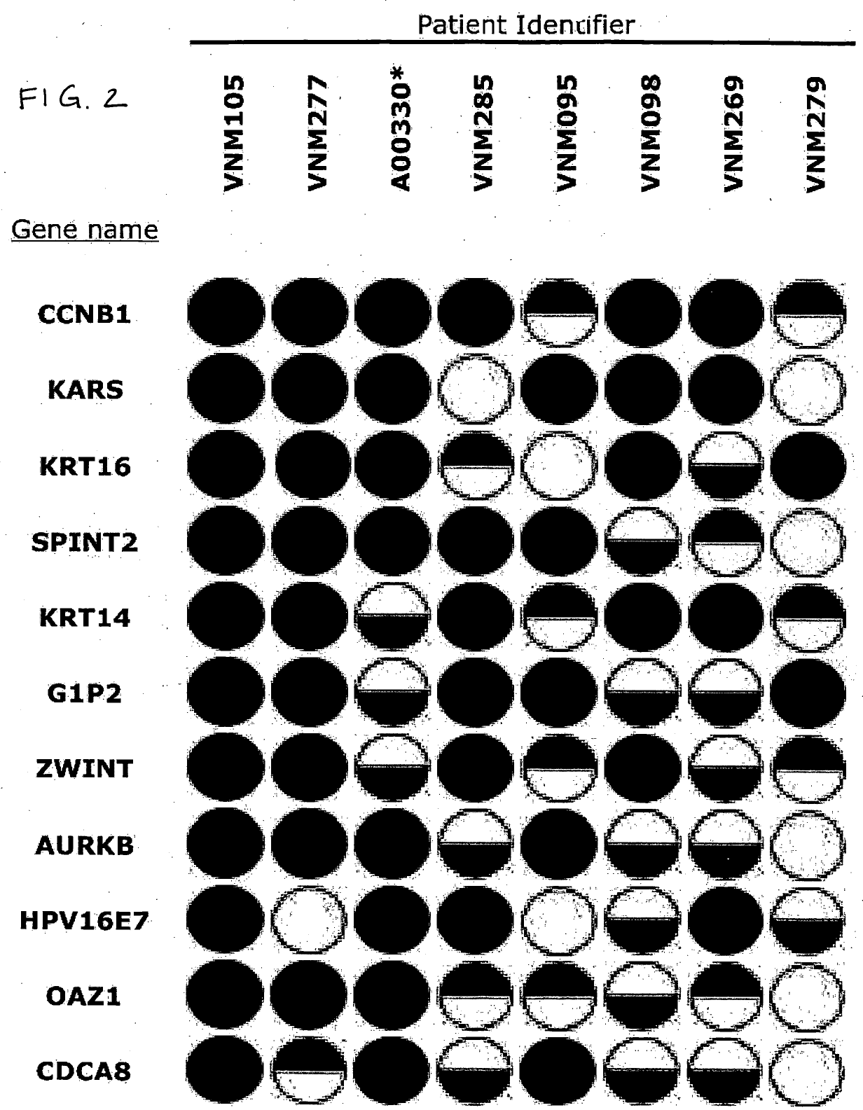

As FIG. 2 shows, DNA macroarray analysis detected transcriptional upregulation in 62 out of 88 patient-gene data points (70.5%). Real-time quantitative RT-PCR detected transcriptional upregulation in 67 of 88 data points (76.1%). Combined, the two methods detect transcriptional upregulation in 79 of 88 data points (89.8%). The two methods agreed with each other in detecting transcriptional upregulation in 59 of 88 data points (67.0%). Occasional disagreement between real-time quantitative RT-PCR and comparative hybridization results has been seen in other studies. The increased sensitivity of real-time quantitative RT-PCR over DNA macroarray analysis accounts for some instances in which real-time quantitative RT-PCR indicated transcriptional upregulation where the macroarray analysis did not. The increased specificity of real-time quantitative RT-PCR over DNA macroarray analysis likely accounts for some instances in which the DNA macroarray indicated transcriptional upregulation where real-time quantitative RT-PCR did not. The primers and probes used in real-time quantitative RT-PCR are gene-specific and thus analyze only one gene of a group of similar genes that may hybridize to a spot on the macroarray. Such cross-hybridization events on the macroarray may indicate transcriptional upregulation of multiple genes in a family of genes with similar sequences. These similar genes could be examined individually using gene-specific primers and probes in real-time quantitative RT-PCR experiments.

Several genes in the set of eleven confirmed genes are known to be upregulated or involved in other cancers. CCNB1 (cyclin B1) is transcriptionally upregulated in several cancers including breast and colon. AURKB (aurora B kinase) is similarly upregulated in a variety of cancers. Changes in SPINT2 (serine protease inhibitor 2) expression have been shown to affect the outcomes of ovarian cancer. OAZ1 (ornithine decarboxylase antizyme 1) is a known tumor suppressor gene. HPV16 E7 (the E7 protein of human papillomavirus 16) is a well-known oncogene for SCCC. As shown in Table 5, HPV16 E7 was detected in the disease specimens from six of the eight patients including the original patient specimen used for RDA. No tests for other HPV genes were performed.

Two other confirmed transcriptionally upregulated genes function in cell division. ZWINT (Zw10 interacting factor) is a kinetochore-associated protein. Because Zw10 is a checkpoint gene, ZWINT may be involved in checkpoint function. The function of CDCA8 (cell division cycle associated protein 8) has not been determined, but it is coexpressed with other cell cycle genes such as CDC2, CDC3, and cyclin.

The other confirmed transcriptionally upregulated genes in this study may be associated with cervical disease. G1P2 (interferon-stimulated protein, 15 kD) is stimulated by interferon, and so may be overproduced as a result of infection. The cervix is relatively susceptible to infection due to its accessibility to the external environment. KRT14 and KRT16 (keratin 14 and keratin 16) are structural proteins that are produced at high levels in the keratinizing squamous epithelium of the cervix. Increased proliferation of tissue that naturally produces keratins is likely to produce increased levels of keratin; such an increase may be reflected at the transcript level.

A recent microarray study examining the transcriptional profiles of several stages of SCCC independently identified two transcriptionally upregulated genes that appear in this study: ARK2/AURKB, which is confirmed here to be transcriptionally upregulated by real-time quantitative RT-PCR, and MYBL2, which appears to be transcriptionally upregulated in four of eight patients by DNA macroarray analysis. MCM2 (minichromosome maintenance protein 2), which is in the same functional family as two other genes identified in the study of Chen et al. (2003) Cancer Res 63:1927-35 (MCM4 and MCM6), is also indicated as transcriptionally upregulated by DNA macroarray analysis. No other genes isolated in this study appear either in the study of Chen et al. or in a microarray study performed in Wong et al. (2003) Clin Cancer Res 2003; 9:5486-92. The genes identified here therefore add key elements to the picture of transcriptionally upregulated genes in SCCC.

In this pilot study, many gene fragments were isolated that are indicated as transciptionally upregulated in both the single patient from which they were isolated and 75% or more of all patients examined by DNA macroarray analysis. While some of these genes such as MCM2, NDRG1, CBR1, and EIF4A have been identified as transcriptional markers of cancer, others such as CALML5 have not been identified as having roles in SCCC or in other cancers.

RDA performed using normal and disease tissues from a single patient identified a panel of 41 genes that was confirmed using amplified cDNA from seven other patients. The genes of interest in the panel are those that have a high correlation of expression in multiple patients. The genes that do not have a high correlation of expression indicate the variable expression that may be a function of differences in neoplastic transformation and/or the growth characteristics of SCCC. One could increase the size of this gene panel by performing RDA on additional SCCC patients and confirming the expression of newly-identified fragments of genes of interest in an expanded number of patients. Panels of genes shown to be transcriptionally upregulated in SCCC, such as those presented in this study, will improve the understanding of this disease and provide the basis for a diagnostic test.

### Example 2

A list of statistically validated genes from five patients using paired (normal vs disease) analysis was prepared. The lists are set forth in Table 5 (upregulated sequences) and Table 6 (down-regulated sequences). The Tables list in the first column the accession number in Genbank; in column 2, the score; and column 3, the fold change in expression (positive or negative). The analysis forward limited the ratio to 2.0 fold, and and 95% confidence as the cut offs.

The statistical analysis was done with an excel plug-in, based on the SAM analysis of Tusher et al. (2001) PNAS Vol 98 no 9.

All of the patients had eight 100 μl tube of amplification for DpnII normal, NlaIII normal, DpnII disease and NlaIII disease. Each 8 tube pool was purified, quantitated and biotinylated using 4×1 μg aliquots. Normal biotinylations were quantitated and equally pooled, disease amplicons were treated similarly.

CEL files from Affymetrix analysis MAS 5 were open with Array Assist version 3.3 and RMA based CHP files were generated. The values for the probe intensities from the RMA CHP files were exported into excel from the visualization module of Array Assist. This allowed statistical analysis of the 5×5 array data with SAM version 2.20.

Patient specimens: Tissue specimens were obtained from ILSBio (Chestertown, Md.) or Genomics Collaborative (Cambridge, Mass.). All patient samples were collected with patient consent in compliance with the company IRBs and with the Code of Federal Regulations (CFR) 45CFR46.101B. All specimens were anonymized by ILS Bio and Genomics Collaborative. Paired squamous cell carcinoma of the cervix (Disease) and non-cancer (Normal) tissues were taken from single patient surgical specimens that had been frozen in liquid nitrogen within 30 minutes of extirpation. Microscope slides were reviewed by a pathologist for diagnosis and staging, and a pathology report was received with each tissue specimen.

RNA isolation: Frozen tissue samples (0.45-1.25 g) were ground to a fine powder under liquid nitrogen. The entire specimen was suspended in 4 ml of room temperature 6 M Guanidine-thiocyanate per 200 mg of tissue. The samples are stored at −80° C. and 4 ml fractions are processed for RNA Isolation. Total RNA was isolated from the 4 ml fractions of the complete tissue resuspension using an RNEasy Midi Kit (Qiagen, Valencia, Calif.). Kit protocols were followed and including the on column DNAse treatment to remove any genomic DNA contamination. Total RNA samples were analyzed using an Agilent (Palo Alto, Calif.) 2100 Bioanalyzer system for 18S and 28S band integrity, quantitated by A280 absorbance, and checked for purity by A260/A280 ratio.

cDNA synthesis: cDNA was synthesized using approx. 5 μg of total RNA from Normal and Disease tissues as template in the Roche (Indianapolis, Iowa) cDNA Synthesis System according to manufacturer's instructions using 2 mM PolyT18_DpnII/NlaIII-V primer (5′-GAGAGTGAGTGATCATGTTTTTTTTTTTTTTTTTTV-3′). Concentrations of Normal and Disease cDNA after the final precipitation were estimated by ethidium bromide dot quantitation with known standards.

Linker Assembly: 10 pmol of each pair of oligos was combined in a final volume of 50 μl (20 mM Tris-HC1 pH: 8.0, 100 mM NaCl), and heated to 95° C. and slow cooled to 4° C. over 3 hours. The DpnII linker for is assembled with: R-BGL-24, sequence 5′-AGCACTCTCCAGCCTCTCACCGCA-3′, and R-BGL-12, sequence 5′-GATCTGCGGTGA-3′) and the NlaIII linker is assembled with: R-BGL-28_NlaIII, sequence 5′-AGCACTCTCCAGCCTCTCACCGCACATG-3′ and R-Bgl-08_NlaIII, sequence 5′-TGCGGTGA-3′).

Amplicon synthesis: Approximately twenty five nanograms of each cDNA sample (Normal and Disease) was digested with DpnII or NlaIII for 90 minutes in an 100 fold excess of enzyme and the appropriate buffer, both reactions were heat killed at 65° C. for 90 minutes and ligated to an excess (5 ug) of appropriate pre-assembled linker for each digest (3 to 12 hrs). Linker and fragmented cDNA ligations were diluted 10 fold with water and used as template for RFA reactions. The yields from two tubes of amplification were used to establish the proper number of cycles and concentration of template for the eight tube experiments. Two identical 100 μl tubes of amplification containing 1.5 μl of the diluted template, 100 pM R-BGL-24 primer, and (final concentration) 66 mM Tris-HC1 pH 8.8 at 25° C., 16 mM (NH4)2SO4, 4 mM MgCl2, 0.2 mM each dNTP. The amplifications were incubated at 72° C. for 3 minutes before the addition of 5 units of Taq polymerase. The 72° C. incubation continued for ten minutes before 24 cycles at 95° C. for 15 seconds and 72° C. for 3 minutes. The yields of amplicon synthesis (A260 nm vs water) were determined for the two-tube amplification after pooling, phenol/chloroform extraction and ethanol precipitation Eight replicate 100 μl amplification reactions as described were performed for each template: DpnII Normal, NlaIII Normal, DpnII Disease and NlaIII Disease. The number of cycles and quantity of template were determined experimentally in the two-tube experiment. Each reaction contained the previously described ingredients and was continued for 24 to 28 cycles, after the initial 10 minutes of incubation.

The DpnII Normal, NlaIII Normal, DpnII Disease and NlaIII Disease amplicons were separately pooled, phenol/chloroform extracted, ethanol precipitated, resuspended in 100 μl TE-1 (1 mM Tris pH 8.0, 0.1 mM EDTA). The RFA amplicon resuspensions were diluted in water and quantitated by A260 and checked for purity by A260/A280 ratio.

Microarray analysis: The microarray analysis of patient 3 combined equal aliquots of biotinylated DpnII Normal and NlaIII Normal amplicons (7.5 μg each, 15 μg total for each array). Disease amplicons were combined similarly. Biotinylated combinations of Normal and Disease amplicons were hybridized to Affymetrix U133A and U133B chipsets (Santa Clara, Calif.). All microarray experiments followed the same hybridization and processing protocols. Biotinylated samples were transferred to the Stanford Protein and Nucleic Acid facility for hybridization to Affymetrix microarrays. The hybridizations, washings and scanning were performed according to the manufacturer's instructions. Image analysis files from the Affymetrix Microarray Analysis Suite 5.1 software (MAS 5.1) were transferred back to our lab for further analysis; some files were generated from the updated software release (GCOS v1.0). ArrayAssist ver. 3.3 (Stratagene, Inc., La Jolla, Calif.) was used to import Affymetrix CEL files and generate intensity values based on the Robust Multi-Array Average (RMA) methods and scatter plots of the RMA derived values. RMA derived intensity values were exported to Microsoft Excel for further statistical characterization.

Replicate microarray analysis: DpnII and NlaIII ligations from a single patient cDNA were pooled for alternate amplicon synthesis protocol. Replicate amplifications were established from the combined cDNA source. 30 tubes of amplification for both Normal and Disease were pooled in groups of six tubes and four groups of 6-tube pool were combined to generate the 24-tube pools. The 6-tube and 24-tube pools (Normal and Disease) were biotinylated in five replicate tubes per sample (Normal and Disease). The 24-tube pools (Normal and Disease) were biotinylated in duplicate. The duplicate biotinylations from the 24-tube Disease labeling were combined (5+5.10 tubes) to generate probe for replicate hybridization results. Biotinylated replicate amplification, replicate biotinylations and sample for replicate hybridization were purified with Mirocon YM-10 centrifugal devices (Millipore, Inc., Billerica, Mass.) and 10 ug was hybridized to Affymetrix U133A plus chips and processed as described. Genes that had intensity values below 100 in the duplicate hybridizations were removed from the list before statistical analysis.

Current settings
Input parameters

| Data type? | Two class paired |
|---|---|
| Arrays centered? | FALSE |
| Delta | 0.619182 |
| Minimum fold change | 2 |
| Test statistic | standard |
| Are data are log scale? | FALSE |
| Number of permutations | 200 |
| Input percentile for exchangeability factor s0 | Automatic choice |
| Number of neighbors for KNN | 10 |
| Seed for Random number generator | 1234567 |

Computed values

| | |
|---|---|
| Estimate of pi0 (proportion of null genes) | 0.860853 |
| Exchangibility factor s0 | 0.097489 |
| s0 percentile | 0.001829 |
| False Discovery Rate (%) | 4.7909 |
| List of Significant Genes for Delta = 0.619 | |

TABLE 5
Up-regulated Genes

| Gene Name | Score(d) | Fold |
|---|---|---|
| gb:AI356412 | 13.29805 | 2.073439 |
| gb:U52144.1 | 11.57092 | 2.221872 |
| gb:BC000323.1 | 11.07087 | 3.681075 |
| gb:BC005807.1 | 10.69444 | 2.577238 |
| gb:AF279900.1 | 10.60844 | 2.338439 |
| gb:M34455.1 | 10.48166 | 2.747295 |
| gb:NM_004526.1 | 10.25601 | 3.355655 |
| gb:BQ021146 | 10.12198 | 3.046884 |
| gb:AK026736.1 | 10.08923 | 4.786415 |
| gb:AA292789 | 10.05358 | 4.071438 |
| gb:AA872583 | 9.470293 | 4.552177 |
| gb:AB032261.1 | 9.296003 | 2.416852 |
| gb:NM_016425.1 | 9.271191 | 3.253378 |
| gb:AB012305.1 | 8.863097 | 2.195054 |
| gb:AA604621 | 8.262645 | 3.021364 |
| gb:NM_004629.1 | 8.148666 | 2.119597 |
| gb:NM_014791.1 | 8.06664 | 4.432061 |
| gb:NM_015895.1 | 7.999641 | 3.100824 |
| gb:BC003186.1 | 7.997644 | 2.318339 |
| gb:BF792864 | 7.955259 | 2.46457 |
| gb:AF030514.1 | 7.895338 | 4.117655 |
| gb:NM_004221.1 | 7.8203 | 2.305959 |
| gb:AA678241 | 7.792887 | 4.178941 |
| gb:AK096921.1 | 7.707892 | 2.186973 |
| gb:NM_001168.1 | 7.636311 | 2.452215 |
| gb:AA807529 | 7.619184 | 2.522812 |
| gb:BC005978.1 | 7.463338 | 3.036774 |
| gb:BF575685 | 7.449131 | 2.022775 |
| gb:NM_014258.1 | 7.435554 | 2.555919 |
| gb:NM_003088.1 | 7.401303 | 2.056414 |
| gb:AA742244 | 7.364581 | 2.326843 |
| gb:NM_002692.1 | 7.332738 | 2.611315 |
| gb:NM_002773.1 | 7.30796 | 7.748466 |
| gb:AU132185 | 7.169148 | 2.812959 |
| gb:NM_003810.1 | 7.162228 | 3.143203 |
| gb:NM_003258.1 | 6.93394 | 3.9068 |
| gb:NM_024057.1 | 6.861549 | 2.260975 |
| gb:NM_004298.1 | 6.856497 | 2.193314 |
| gb:NM_005733.1 | 6.825721 | 5.188451 |
| gb:NM_002497.1 | 6.799315 | 2.053293 |
| gb:NM_004111.3 | 6.74969 | 4.453379 |
| gb:BC007986.1 | 6.628188 | 3.763444 |
| gb:NM_007370.1 | 6.511454 | 2.354079 |
| gb:NM_004856.3 | 6.497579 | 2.684124 |
| gb:NM_001826.1 | 6.288204 | 2.588918 |
| gb:AA496034 | 6.274703 | 8.54853 |
| gb:NM_005496.1 | 6.268959 | 3.537487 |
| gb:NM_018098.1 | 6.22581 | 9.416711 |
| gb:U25975.1 | 6.117543 | 2.919481 |
| gb:AL162069.1 | 6.116012 | 2.141699 |
| gb:NM_003090.1 | 6.0845 | 2.034508 |
| gb:AF116616.1 | 6.083798 | 2.349773 |
| gb:AI859865 | 6.063413 | 4.102921 |
| gb:AY029179.1 | 6.062696 | 5.311577 |
| gb:NM_004701.2 | 6.030892 | 6.924434 |
| gb:NM_002875.1 | 6.0035 | 4.85414 |
| gb:L20817.1 | 5.977154 | 2.13731 |
| gb:AF154005.1 | 5.963634 | 2.933462 |
| gb:AF027205.1 | 5.961738 | 3.039129 |
| gb:AB032931.1 | 5.94993 | 3.597274 |
| gb:NM_021953.1 | 5.888388 | 2.163775 |
| gb:NM_018565.1 | 5.833329 | 3.508796 |
| gb:BC000192.1 | 5.801855 | 2.210916 |
| gb:BE407516 | 5.791029 | 5.936745 |
| gb:NM_002131.1 | 5.788401 | 3.077765 |
| gb:AI335263 | 5.75311 | 5.494922 |
| gb:AI671049 | 5.726918 | 2.129483 |
| gb:NM_017760.1 | 5.712886 | 2.160046 |
| gb:BC002493.1 | 5.709131 | 2.108892 |
| gb:U57059.1 | 5.683508 | 2.739841 |
| gb:BC001068.1 | 5.675421 | 4.085797 |
| gb:NM_024053.1 | 5.615715 | 2.023736 |
| gb:AF155827.1 | 5.591736 | 3.463826 |
| gb:NM_018186.1 | 5.574941 | 2.625084 |
| gb:BE326728 | 5.568252 | 2.077951 |
| gb:AB011446.1 | 5.565719 | 2.041286 |
| gb:AF132203.1 | 5.534002 | 2.17255 |
| gb:NM_007295.1 | 5.529633 | 2.078673 |
| gb:AW444761 | 5.529324 | 4.866878 |
| gb:NM_016445.1 | 5.495657 | 9.243172 |
| gb:L11315.1 | 5.43426 | 2.226852 |
| gb:AL117600.1 | 5.422221 | 2.541498 |
| gb:AA496034 | 5.370579 | 4.128394 |
| gb:BE965369 | 5.361841 | 3.122791 |
| gb:AI147467 | 5.342111 | 2.370368 |
| gb:NM_002794.1 | 5.341835 | 2.092086 |
| gb:NM_001954.2 | 5.254373 | 2.279717 |
| gb:AK025578.1 | 5.24602 | 6.581174 |
| gb:BC002551.1 | 5.241209 | 2.0418 |
| gb:NM_001827.1 | 5.209715 | 6.453837 |
| gb:BC001651.1 | 5.208849 | 3.192785 |
| gb:NM_017975.1 | 5.2084 | 3.170313 |
| gb:NM_001070.1 | 5.180165 | 2.285201 |
| gb:NM_003659.1 | 5.176255 | 2.348269 |
| gb:AU149868 | 5.150811 | 2.252701 |
| gb:AA143765 | 5.146559 | 2.506182 |
| gb:AI139629 | 5.136266 | 2.245211 |
| gb:AF321125.1 | 5.133802 | 2.991579 |
| gb:NM_005030.1 | 5.083826 | 4.395281 |
| U48705 | 5.083138 | 2.272494 |
| gb:NM_001761.1 | 5.076935 | 2.207289 |
| gb:BC000025.1 | 5.076782 | 2.074374 |
| gb:NM_014078.1 | 5.069001 | 2.38177 |
| gb:NM_001255.1 | 5.043577 | 5.316664 |
| gb:AJ011712 | 5.005358 | 4.504907 |
| gb:NM_005915.2 | 5.003984 | 2.265817 |
| Cluster Incl. | 4.964007 | 2.205853 |
| gb:AI659477 | 4.958067 | 2.381157 |
| gb:AL520675 | 4.935904 | 3.091951 |
| gb:AY028916.1 | 4.930758 | 2.598956 |
| gb:NM_024037.1 | 4.920929 | 2.14717 |
| gb:NM_002829.1 | 4.890193 | 2.168488 |
| gb:BE502436 | 4.880352 | 2.8404 |
| gb:AF112857.1 | 4.862581 | 2.859888 |
| gb:NM_021067.1 | 4.854889 | 5.649078 |
| gb:AF116682.1 | 4.854241 | 2.445525 |
| gb:BE614567 | 4.843176 | 2.422848 |
| gb:M23114.1 | 4.778785 | 2.343331 |
| gb:BC002842.1 | 4.774779 | 2.979006 |
| gb:NM_003523.1 | 4.753338 | 2.284203 |
| gb:M63310.1 | 4.732965 | 4.096912 |
| gb:AW629423 | 4.718609 | 3.479637 |
| gb:BE999972 | 4.698615 | 2.449703 |
| gb:NM_020188.1 | 4.692425 | 2.24172 |
| gb:NM_014708.1 | 4.691541 | 2.733665 |
| gb:BF796470 | 4.689511 | 2.572188 |
| gb:NM_002589.1 | 4.67183 | 2.25079 |
| gb:BE793789 | 4.62658 | 3.422382 |
| gb:AF191495.1 | 4.58612 | 2.772018 |
| gb:L23982 | 4.562762 | 2.237334 |
| gb:NM_022873.1 | 4.553791 | 2.810733 |
| gb:BG499974 | 4.544529 | 2.169865 |
| gb:AI925583 | 4.535102 | 2.019603 |
| gb:AA824298 | 4.485533 | 3.438127 |
| gb:BC002556.1 | 4.477363 | 2.665134 |
| gb:U19348.1 | 4.475593 | 2.290256 |
| gb:AF070448.1 | 4.450265 | 4.11179 |
| gb:NM_003389.1 | 4.446975 | 2.329624 |
| gb:AL117466.1 | 4.442089 | 2.104741 |
| gb:NM_003733.1 | 4.441812 | 2.353377 |
| gb:NM_022346.1 | 4.44144 | 3.658814 |
| gb:AI828018 | 4.436728 | 3.717337 |
| gb:AI458313 | 4.422255 | 2.038574 |
| gb:AA648913 | 4.414655 | 2.003626 |
| gb:NM_003234.1 | 4.411416 | 2.183241 |
| gb:AI700633 | 4.401964 | 2.280244 |
| gb:AF060925.1 | 4.399041 | 2.251777 |
| gb:NM_003878.1 | 4.395727 | 2.43544 |
| gb:NM_001645.2 | 4.388418 | 3.106617 |
| gb:NM_006397.1 | 4.374545 | 2.970192 |
| gb:NM_000888.3 | 4.35942 | 3.029661 |
| gb:NM_001569.2 | 4.359388 | 2.324055 |
| gb:NM_016448.1 | 4.355656 | 4.418633 |
| gb:BE644935 | 4.35025 | 2.206349 |
| gb:NM_002201.2 | 4.339679 | 2.475086 |
| gb:NM_001809.2 | 4.335798 | 2.600309 |
| gb:BG230614 | 4.335158 | 2.235015 |
| gb:AF172398.2 | 4.312662 | 2.966583 |
| gb:BG029496 | 4.307067 | 2.255424 |
| gb:Z25521.1 | 4.303515 | 2.011971 |
| gb:NM_001211.2 | 4.296818 | 2.942222 |
| gb:NM_004817.1 | 4.295354 | 2.736985 |
| gb:AL554008 | 4.285281 | 2.599522 |
| gb:AK024274.1 | 4.285061 | 2.053843 |
| gb:D26351.1 | 4.247083 | 2.393829 |
| gb:NM_004219.2 | 4.247016 | 5.869898 |
| gb:NM_003710.1 | 4.230839 | 3.504394 |
| gb:BF059136 | 4.211477 | 2.155769 |
| gb:AI076810 | 4.198071 | 2.10749 |
| gb:BF031829 | 4.192669 | 13.87874 |
| gb:AL561834 | 4.187777 | 4.014664 |
| gb:AL512737.1 | 4.163687 | 2.040162 |
| gb:NM_018265.1 | 4.162218 | 5.294019 |
| gb:BE966236 | 4.153452 | 8.765389 |
| gb:W46388 | 4.147097 | 3.250984 |
| gb:BG170541 | 4.147064 | 2.541386 |
| gb:AW138734 | 4.146493 | 2.785771 |
| gb:AA749427 | 4.088583 | 3.207072 |
| gb:BF111870 | 4.085834 | 2.662922 |
| gb:U90236.2 | 4.068831 | 2.154903 |
| gb:NM_022443.1 | 4.066857 | 2.361269 |
| gb:NM_022127.1 | 4.06674 | 2.680992 |
| gb:N58493 | 4.065472 | 2.563552 |
| gb:L25541.1 | 4.062016 | 4.471281 |
| gb:NM_016185.1 | 4.060718 | 3.245677 |
| gb:BF343007 | 4.054292 | 6.47455 |
| gb:N57927 | 4.025394 | 2.371832 |
| gb:BF001806 | 4.017325 | 2.536594 |
| gb:AL117612.1 | 4.005722 | 9.976439 |
| gb:AW241779 | 3.992105 | 2.001442 |
| gb:NM_021199.1 | 3.986566 | 2.41813 |
| gb:NM_000540.1 | 3.985975 | 3.459618 |
| gb:NM_001216.1 | 3.969115 | 2.071194 |
| gb:BC004396.1 | 3.968834 | 3.699702 |
| gb:AF348143.1 | 3.956325 | 2.196841 |
| gb:AV733308 | 3.952912 | 2.001243 |
| gb:NM_002658.1 | 3.948435 | 3.247171 |
| gb:NM_005196.1 | 3.944576 | 4.043725 |
| gb:AI610869 | 3.941297 | 2.273906 |
| gb:K03226.1 | 3.933151 | 3.93393 |
| gb:NM_017445.1 | 3.921824 | 2.600844 |
| gb:AF116624.1 | 3.915643 | 2.243109 |
| gb:AB040903.1 | 3.902061 | 2.080919 |
| gb:NM_002592.1 | 3.900205 | 3.382869 |
| gb:BE379761 | 3.878026 | 2.292507 |
| gb:AI246687 | 3.868859 | 2.18667 |
| gb:NM_014109.1 | 3.866323 | 4.644188 |
| gb:BE614410 | 3.864824 | 3.218068 |
| gb:AW005535 | 3.84866 | 3.013424 |
| gb:W79394 | 3.842651 | 2.526993 |
| gb:BE961977 | 3.833426 | 3.894171 |
| gb:AF098158.1 | 3.83019 | 3.248849 |
| gb:NM_004456.1 | 3.810443 | 4.033627 |
| gb:BC000903.1 | 3.8073 | 2.031509 |
| gb:NM_001565.1 | 3.804563 | 10.73768 |
| gb:AW014155 | 3.803083 | 2.066669 |
| gb:NM_003686.1 | 3.802051 | 2.563647 |
| gb:AL524035 | 3.799066 | 4.150481 |
| gb:AA632295 | 3.797389 | 4.333025 |
| gb:BC000676.1 | 3.793205 | 4.048473 |
| gb:BF590021 | 3.792872 | 2.264617 |
| gb:AL162742 | 3.790673 | 2.606338 |
| gb:NM_002276.1 | 3.786189 | 7.706584 |
| gb:NM_003220.1 | 3.75986 | 2.145396 |
| gb:NM_024945.1 | 3.759777 | 3.464741 |
| gb:AK021779.1 | 3.752441 | 2.146131 |
| gb:AF001294.1 | 3.751168 | 3.182428 |
| gb:AI859865 | 3.750806 | 3.492229 |
| gb:NM_001274.1 | 3.748193 | 3.997267 |
| gb:NM_019618.1 | 3.74425 | 3.110211 |
| gb:AI825926 | 3.743313 | 2.029889 |
| http://genome- | 3.738495 | 2.639172 |
| gb:NM_001254.1 | 3.726321 | 3.188656 |
| gb:BG291649 | 3.725706 | 3.781371 |
| gb:NM_016359.1 | 3.724348 | 8.299707 |
| gb:NM_004184.2 | 3.708993 | 2.34874 |
| gb:NM_017793.1 | 3.688253 | 2.498112 |
| gb:NM_005682.1 | 3.68666 | 2.507401 |
| gb:NM_015366.1 | 3.672665 | 2.823743 |
| gb:NM_006332.1 | 3.66376 | 2.213443 |
| gb:AF269167.1 | 3.663001 | 2.651062 |
| gb:BE670097 | 3.659231 | 2.023043 |
| gb:NM_006384.2 | 3.658043 | 2.184702 |
| gb:Z19574 | 3.655948 | 6.92487 |
| gb:NM_002674.1 | 3.655027 | 2.927281 |
| gb:NM_024430.1 | 3.653963 | 2.238951 |
| gb:AI709055 | 3.650084 | 2.526685 |
| gb:AK023368.1 | 3.646452 | 2.350113 |
| gb:AA723810 | 3.630581 | 15.30752 |
| gb:AA371513 | 3.627807 | 2.153735 |
| gb:AI674647 | 3.625412 | 2.02814 |
| gb:AK023411.1 | 3.613633 | 2.253714 |
| gb:D88357.1 | 3.612109 | 2.693981 |
| gb:AW250952 | 3.610697 | 2.173627 |
| gb:NM_002534.1 | 3.608906 | 3.673515 |
| gb:NM_001943.1 | 3.598368 | 8.678741 |
| gb:NM_005101.1 | 3.598347 | 2.227004 |
| gb:NM_018685.1 | 3.595804 | 4.057398 |
| gb:NM_018131.1 | 3.588066 | 11.39532 |
| gb:D84476.1 | 3.580064 | 2.02579 |
| gb:NM_006636.2 | 3.580045 | 2.925523 |
| gb:AL515918 | 3.578888 | 2.189243 |
| gb:NM_022116.1 | 3.573244 | 2.056324 |
| gb:AU159942 | 3.564615 | 9.169177 |
| gb:NM_000422.1 | 3.555886 | 6.819015 |
| gb:NM_004725.1 | 3.555102 | 2.141084 |
| gb:NM_015455.1 | 3.543992 | 2.146768 |
| gb:NM_006444.1 | 3.540988 | 3.522271 |
| gb:NM_005084.1 | 3.53471 | 2.378701 |
| gb:AL136659.1 | 3.534205 | 2.135216 |
| Cluster Incl. | 3.52887 | 2.207096 |
| gb:AL049709 | 3.526154 | 2.027843 |
| gb:BG492359 | 3.514775 | 4.499805 |
| gb:W74442 | 3.507818 | 2.279507 |
| gb:AI656610 | 3.504962 | 2.203946 |
| gb:NM_018455.1 | 3.488891 | 3.317529 |
| gb:AL137654.1 | 3.488282 | 3.146043 |
| gb:BE870509 | 3.485352 | 2.053901 |
| gb:NM_031299.1 | 3.48227 | 2.169485 |
| gb:NM_003158.1 | 3.481027 | 4.38005 |
| gb:BC002506.1 | 3.480018 | 2.195893 |
| gb:BC000893.1 | 3.476814 | 2.381096 |
| gb:NM_007027.1 | 3.474079 | 2.754272 |
| gb:U88968.1 | 3.469773 | 2.195582 |
| gb:U38945.1 | 3.468269 | 3.756643 |
| gb:AF043294.2 | 3.459507 | 2.20479 |
| gb:AK001261.1 | 3.45398 | 3.464476 |
| gb:AL523965 | 3.452653 | 2.047059 |
| gb:NM_003981.1 | 3.450659 | 4.611305 |
| gb:BG338983 | 3.450116 | 2.231229 |
| gb:AI692974 | 3.439571 | 2.116174 |
| gb:AF047002.1 | 3.437225 | 2.220861 |
| gb:NM_002953.1 | 3.427075 | 2.121411 |
| gb:AJ249377.1 | 3.424068 | 3.145393 |
| gb:AW662246 | 3.412959 | 6.637305 |
| gb:AA502912 | 3.407376 | 2.035724 |
| gb:BF439522 | 3.406509 | 2.989133 |
| gb:NM_002916.1 | 3.397521 | 3.72511 |
| gb:M80927.1 | 3.390842 | 7.491546 |
| gb:BC000433.1 | 3.390617 | 6.100624 |
| gb:BC001188.1 | 3.389422 | 6.352186 |
| gb:NM_015515.1 | 3.384232 | 2.253562 |
| gb:BC005264.1 | 3.383215 | 2.151196 |
| gb:BE646208 | 3.363535 | 3.410127 |
| gb:NM_006187.1 | 3.346988 | 2.524708 |
| gb:AF043337.1 | 3.346466 | 2.582312 |
| gb:AU152107 | 3.341949 | 3.746381 |
| gb:BC001886.1 | 3.339914 | 5.064891 |
| gb:AI739071 | 3.338081 | 2.349266 |
| gb:AF227192.1 | 3.330695 | 2.030116 |
| gb:AF285120.1 | 3.329302 | 3.327612 |
| gb:AW003367 | 3.319722 | 2.365608 |
| gb:NM_002358.2 | 3.317769 | 2.602221 |
| gb:NM_003600.1 | 3.316273 | 4.500754 |
| gb:BC000433.1 | 3.312577 | 3.649806 |
| gb:NM_001710.1 | 3.306534 | 3.79272 |
| gb:NM_024629.1 | 3.29473 | 5.905641 |
| gb:BF673013 | 3.287239 | 2.00331 |
| gb:AI949095 | 3.286588 | 2.017345 |
| gb:NM_005940.2 | 3.282639 | 5.654568 |
| gb:M80927.1 | 3.281289 | 3.767399 |
| gb:NM_022346.1 | 3.265867 | 2.120061 |
| gb:AK000529.1 | 3.261861 | 3.027674 |
| gb:NM_003318.1 | 3.260255 | 4.129195 |
| gb:AL035689 | 3.243517 | 2.54988 |
| gb:AF047473.1 | 3.23848 | 2.050973 |
| gb:NM_017915.1 | 3.231377 | 2.538028 |
| gb:BE872974 | 3.228551 | 2.131186 |
| gb:U36310.1 | 3.227895 | 2.542747 |
| gb:AI631846 | 3.227368 | 6.064568 |
| gb:AI348935 | 3.215377 | 2.456443 |
| gb:NM_001071.1 | 3.212543 | 2.366236 |
| gb:AA772285 | 3.200966 | 2.480139 |
| gb:AI193090 | 3.19294 | 2.172871 |
| gb:AI857685 | 3.189226 | 2.700681 |
| gb:NM_001690.1 | 3.182463 | 2.51066 |
| gb:NM_014479.1 | 3.17955 | 2.245565 |
| gb:AI765051 | 3.178089 | 2.082429 |
| gb:NM_005498.2 | 3.177362 | 3.53494 |
| gb:NM_000946.1 | 3.171891 | 2.622423 |
| gb:BF431260 | 3.170642 | 2.529241 |
| gb:NM_013441.1 | 3.167097 | 2.26255 |
| gb:BC005238.1 | 3.166171 | 3.004353 |
| gb:NM_002662.1 | 3.165508 | 2.293975 |
| gb:NM_014398.1 | 3.159875 | 6.13096 |
| gb:AV702405 | 3.152254 | 2.70962 |
| gb:NM_016332.1 | 3.148815 | 2.332385 |
| gb:AW299700 | 3.145081 | 2.273753 |
| gb:NM_014501.1 | 3.136968 | 3.405215 |
| gb:NM_018087.1 | 3.134509 | 2.192505 |
| gb:AU147044 | 3.12874 | 2.386472 |
| gb:NM_000224.1 | 3.124938 | 5.315012 |
| gb:NM_005242.2 | 3.124793 | 2.181692 |
| gb:AJ249377.1 | 3.117141 | 5.245602 |
| gb:NM_020242.1 | 3.115917 | 3.545652 |
| gb:NM_003681.1 | 3.114564 | 2.914522 |
| gb:BE966748 | 3.11157 | 2.527484 |
| gb:NM_007274.1 | 3.10364 | 2.196363 |
| gb:J03189.1 | 3.102198 | 3.868634 |
| gb:NM_019114.1 | 3.091107 | 3.600993 |
| gb:BF060747 | 3.090644 | 2.230727 |
| gb:NM_030769.1 | 3.080538 | 2.71293 |
| gb:BC023566.1 | 3.078348 | 4.753038 |
| gb:BG403615 | 3.074631 | 2.007102 |
| gb:NM_018846.1 | 3.073295 | 2.020406 |
| gb:BC001872.1 | 3.070513 | 3.932101 |
| gb:AL525412 | 3.068096 | 2.026846 |
| gb:NM_003158.1 | 3.059353 | 6.823038 |
| gb:AW006750 | 3.048386 | 2.653255 |
| gb:D84143.1 | 3.043701 | 2.58387 |
| gb:D31661.1 | 3.039567 | 2.239618 |
| gb:AI339710 | 3.03435 | 3.123205 |
| gb:Z25425.1 | 3.033931 | 2.924178 |
| gb:D84140.1 | 3.031436 | 2.252882 |
| gb:N73742 | 3.029841 | 2.331927 |
| gb:AW471176 | 3.027816 | 3.44903 |
| gb:D84143.1 | 3.025668 | 3.720737 |
| gb:NM_016817.1 | 3.014845 | 3.653319 |
| gb:AW449022 | 3.011806 | 2.050443 |
| gb:NM_012310.2 | 3.008515 | 3.104513 |
| gb:AI601101 | 3.006924 | 2.842567 |
| gb:AW151538 | 3.005017 | 2.22991 |
| gb:NM_003035.1 | 2.998296 | 2.007723 |
| gb:AF232674.1 | 2.998056 | 2.931658 |
| gb:NM_024729.1 | 2.995435 | 3.590425 |
| gb:BC042986.1 | 2.990801 | 8.677078 |
| gb:NM_018092.1 | 2.985077 | 2.248528 |
| gb:NM_002573.1 | 2.984694 | 2.039016 |
| gb:NM_006342.1 | 2.981719 | 2.794855 |
| gb:NM_000270.1 | 2.978858 | 2.647962 |
| gb:BC004908.1 | 2.972265 | 2.65699 |
| gb:NM_007019.1 | 2.971466 | 2.050254 |
| gb:AW075105 | 2.971053 | 4.151862 |
| gb:M83248.1 | 2.965507 | 2.503584 |
| gb:NM_016426.1 | 2.964443 | 3.139644 |
| gb:BG170335 | 2.962479 | 4.478583 |
| gb:AL353759 | 2.960285 | 2.207842 |
| gb:AI479075 | 2.959739 | 5.854275 |
| gb:AW009562 | 2.956042 | 2.067087 |
| gb:NM_001813.1 | 2.954651 | 2.500027 |
| gb:N20927 | 2.950545 | 2.337778 |
| gb:AV712602 | 2.945519 | 2.166466 |
| gb:BE672260 | 2.943223 | 2.323358 |
| gb:BC004863.1 | 2.932317 | 6.83904 |
| gb:AL133260 | 2.932249 | 3.128585 |
| gb:X15132.1 | 2.931174 | 3.397109 |
| gb:M24668.1 | 2.926102 | 2.650092 |
| gb:NM_005245.1 | 2.924991 | 2.052154 |
| gb:NM_018454.1 | 2.921255 | 3.9794 |
| gb:NM_000101.1 | 2.915537 | 2.753024 |
| gb:AI357639 | 2.912192 | 2.531759 |
| gb:AA904259 | 2.906959 | 2.608729 |
| gb:NM_004350.1 | 2.900878 | 2.552887 |
| Cluster Incl. | 2.900837 | 2.96234 |
| gb:NM_004833.1 | 2.896441 | 3.629611 |
| gb:BE220003 | 2.896237 | 2.288414 |
| gb:BF382281 | 2.885162 | 2.507062 |
| gb:NM_018154.1 | 2.883585 | 2.690747 |
| gb:AL545105 | 2.879497 | 2.142777 |
| gb:AI830823 | 2.876693 | 2.578443 |
| gb:NM_030755.1 | 2.876106 | 2.030953 |
| gb:NM_004360.1 | 2.868729 | 6.965515 |
| gb:AA909765 | 2.868437 | 2.554337 |
| gb:AF101051.1 | 2.859085 | 2.90133 |
| gb:NM_017697.1 | 2.849649 | 8.05511 |
| gb:AI761713 | 2.846964 | 5.471746 |
| gb:NM_005613.2 | 2.846446 | 2.279577 |
| Cluster Incl. | 2.845372 | 3.053762 |
| gb:AI650364 | 2.839137 | 2.246231 |
| gb:BF680458 | 2.837485 | 3.412115 |
| gb:AK023669.1 | 2.835286 | 2.245081 |
| Cluster Incl. | 2.834666 | 3.592135 |
| gb:NM_006461.1 | 2.82726 | 2.471186 |
| gb:BG288007 | 2.811898 | 2.386329 |
| gb:AJ242547.1 | 2.809156 | 3.878585 |
| gb:AI761561 | 2.808296 | 3.177361 |
| gb:AL136877.1 | 2.807217 | 3.58683 |
| gb:AI346350 | 2.806486 | 2.56825 |
| gb:NM_012485.1 | 2.804766 | 3.683997 |
| gb:N32557 | 2.800462 | 3.643227 |
| gb:NM_005729.1 | 2.797847 | 2.541191 |
| gb:AI659020 | 2.790977 | 2.163365 |
| gb:NM_014278.1 | 2.786025 | 2.014526 |
| gb:NM_017767.1 | 2.775282 | 3.244919 |
| gb:AF342816.1 | 2.77137 | 2.263962 |
| gb:NM_002691.1 | 2.770556 | 2.009682 |
| gb:NM_000269.1 | 2.766034 | 2.436716 |
| gb:NM_005562.1 | 2.7466 | 2.268642 |
| gb:L08599.1 | 2.746161 | 3.461623 |
| gb:NM_002997.1 | 2.743164 | 6.010365 |
| gb:AI382146 | 2.742882 | 3.172764 |
| gb:AB037784.1 | 2.738166 | 5.816846 |
| gb:NM_018944.1 | 2.737228 | 2.088877 |
| gb:AL121975 | 2.734052 | 2.185504 |
| gb:NM_002356.4 | 2.729413 | 2.202127 |
| gb:AF225416.1 | 2.722203 | 2.092547 |
| gb:M94363 | 2.712392 | 2.036 |
| gb:AW269645 | 2.710505 | 6.180277 |
| gb:AK026105.1 | 2.708074 | 2.00199 |
| gb:NM_025111.1 | 2.706847 | 2.42177 |
| gb:AA960844 | 2.706704 | 2.164741 |
| gb:AW025572 | 2.706273 | 3.061587 |
| gb:AI742057 | 2.704638 | 2.313108 |
| gb:NM_014736.1 | 2.703721 | 8.008323 |
| gb:U94592.1 | 2.70141 | 2.550038 |
| gb:NM_018123.1 | 2.701147 | 8.853151 |
| gb:NM_003447.1 | 2.69458 | 2.736082 |
| U38545 | 2.683425 | 2.838143 |
| gb:AA129612 | 2.682989 | 2.37103 |
| gb:NM_021101.1 | 2.678999 | 3.417188 |
| gb:AF043583.1 | 2.676234 | 2.596985 |
| gb:NM_005764.2 | 2.672318 | 2.474693 |
| gb:AL137725.1 | 2.670943 | 2.32113 |
| gb:NM_005700.1 | 2.668976 | 2.0974 |
| Cluster Incl. | 2.668371 | 4.313234 |
| gb:BE045993 | 2.663439 | 2.989235 |
| gb:NM_004226.1 | 2.655357 | 2.32265 |
| gb:NM_005402.1 | 2.652426 | 2.915894 |
| gb:NM_016619.1 | 2.648568 | 6.03445 |
| gb:NM_022842.1 | 2.648017 | 2.276917 |
| gb:AF314544.1 | 2.646102 | 2.023338 |
| gb:AF213033.1 | 2.6437 | 2.989368 |
| gb:AI924134 | 2.642681 | 2.244864 |
| gb:NM_004301.1 | 2.639214 | 3.090718 |
| gb:U63743.1 | 2.639113 | 4.870141 |
| gb:NM_002456.1 | 2.637636 | 3.818249 |
| gb:AI830823 | 2.632873 | 2.081439 |
| gb:L25259.1 | 2.63218 | 2.858215 |
| gb:U05598.1 | 2.630459 | 2.77693 |
| gb:U82819.1 | 2.62674 | 2.404118 |
| gb:NM_001559.1 | 2.626419 | 2.947739 |
| gb:AW269686 | 2.625405 | 2.125437 |
| gb:AL118502 | 2.623799 | 2.235237 |
| gb:AF043583.1 | 2.62308 | 2.081606 |
| gb:AW473802 | 2.61968 | 2.053686 |
| gb:AU153848 | 2.618396 | 5.871507 |
| gb:U29343.1 | 2.615907 | 5.644847 |
| gb:NM_022776.1 | 2.615492 | 2.198048 |
| gb:AB037780.1 | 2.604678 | 2.207944 |
| gb:AV697515 | 2.597991 | 3.099258 |
| gb:BC002439.1 | 2.597078 | 2.923332 |
| gb:AK000049.1 | 2.595313 | 2.697729 |
| gb:NM_006527.1 | 2.593202 | 2.714585 |
| gb:NM_001237.1 | 2.59234 | 3.012972 |
| gb:NM_025236.1 | 2.587272 | 3.64236 |
| gb:NM_005342.1 | 2.586065 | 3.87799 |
| gb:NM_031217.1 | 2.585023 | 3.702286 |
| gb:NM_016391.1 | 2.582407 | 2.026219 |
| gb:NM_006101.1 | 2.57888 | 2.336981 |
| gb:NM_004532.1 | 2.573199 | 4.303386 |
| gb:AF043584.1 | 2.570487 | 2.049736 |
| gb:AF394735.1 | 2.566942 | 3.15012 |
| gb:D17218.1 | 2.560475 | 2.061026 |
| Cluster Incl. | 2.552579 | 2.584911 |
| gb:BC005170.1 | 2.546736 | 2.424156 |
| gb:AI203028 | 2.546073 | 2.175007 |
| gb:NM_153838.1 | 2.545734 | 2.233998 |
| gb:BF001941 | 2.537222 | 11.82098 |
| gb:AL518328 | 2.521886 | 2.363447 |
| gb:AV691491 | 2.521807 | 3.17084 |
| gb:AF317417.1 | 2.520692 | 2.086873 |
| gb:NM_020990.2 | 2.512714 | 4.378744 |
| gb:BE000929 | 2.509427 | 2.122939 |
| gb:AA020826 | 2.503581 | 2.618892 |
| gb:NM_001530.1 | 2.499941 | 2.360007 |
| http://genome- | 2.496563 | 2.83913 |
| gb:AJ276691.1 | 2.496008 | 2.132651 |
| gb:NM_006456.1 | 2.493553 | 2.64988 |
| gb:T90295 | 2.489638 | 2.510323 |
| gb:L21961.1 | 2.482257 | 2.657646 |
| gb:NM_004994.1 | 2.480699 | 5.518491 |
| gb:BF242905 | 2.48001 | 2.963104 |
| gb:NM_018492.1 | 2.477877 | 4.180322 |
| gb:NM_002422.2 | 2.477647 | 9.465921 |
| gb:NM_005764.1 | 2.475209 | 3.477412 |
| gb:NM_003920.1 | 2.472737 | 2.940525 |
| gb:X02189 | 2.472448 | 2.00547 |
| gb:NM_015925.1 | 2.469574 | 2.552017 |
| gb:NM_000077.1 | 2.460527 | 5.842632 |
| gb:NM_017423.1 | 2.460096 | 2.175606 |
| gb:AV681807 | 2.457284 | 2.932434 |
| gb:AB018009.1 | 2.454704 | 5.936066 |
| gb:NM_018214.1 | 2.453823 | 2.079589 |
| gb:NM_022096.1 | 2.453718 | 2.435727 |
| gb:U77949.1 | 2.452417 | 4.916473 |
| gb:AK026133.1 | 2.450305 | 2.566736 |
| gb:NM_017699.1 | 2.448017 | 2.648431 |
| gb:BE645551 | 2.442985 | 2.333033 |
| gb:NM_001723.1 | 2.440243 | 16.60243 |
| gb:NM_006410.1 | 2.438434 | 2.264018 |
| gb:NM_014750.1 | 2.432857 | 3.601775 |
| gb:NM_005564.1 | 2.432375 | 6.538428 |
| gb:BF444916 | 2.430871 | 2.709049 |
| gb:NM_005980.1 | 2.428975 | 9.293755 |
| gb:NM_031308.1 | 2.426285 | 2.563486 |
| gb:AI935915 | 2.424671 | 2.7622 |
| gb:X93006.1 | 2.422348 | 2.094593 |
| gb:AJ225092.1 | 2.41994 | 2.785241 |
| gb:NM_004207.1 | 2.413141 | 4.202063 |
| gb:AF228422.1 | 2.411265 | 5.38812 |
| gb:NM_024915.1 | 2.409076 | 2.044325 |
| gb:NM_000346.1 | 2.404471 | 2.169194 |

TABLE 6
Down-regulated genes

| Gene Name | Score(d) | Fold |
|---|---|---|
| gb:NM_004166.1 | −19.3916 | 0.25944 |
| gb:AK002203.1 | −16.8099 | 0.32739 |
| gb:AF063002.1 | −12.3823 | 0.16967 |
| gb:NM_000125.1 | −11.2997 | 0.18002 |
| gb:AF220153.1 | −10.9847 | 0.20320 |
| gb:AW193698 | −10.0098 | 0.31843 |
| gb:U19495.1 | −9.82527 | 0.39614 |
| gb:NM_002380.2 | −9.49871 | 0.20657 |
| gb:U29538.1 | −9.1227 | 0.23184 |
| gb:M57399.1 | −9.0765 | 0.38867 |
| gb:AL021786 | −8.78038 | 0.25735 |
| gb:NM_021219.1 | −8.42909 | 0.29565 |
| gb:NM_004867.1 | −8.33316 | 0.31348 |
| gb:NM_001449.1 | −8.12524 | 0.10127 |
| gb:NM_022159.1 | −8.03579 | 0.37874 |
| gb:AL050264.1 | −7.83465 | 0.33698 |
| gb:NM_004684.1 | −7.5319 | 0.33210 |
| gb:AA702701 | −7.50594 | 0.46576 |
| gb:NM_002599.1 | −7.46674 | 0.39794 |
| gb:AA025351 | −7.39204 | 0.34428 |
| gb:NM_003430.1 | −7.37494 | 0.32835 |
| gb:AI659927 | −7.21724 | 0.35366 |
| gb:BF739943 | −7.12968 | 0.47177 |
| gb:AL518391 | −7.10022 | 0.23619 |
| gb:M95585.1 | −6.88688 | 0.48873 |
| gb:AB000889.1 | −6.82976 | 0.25666 |
| gb:NM_003005.2 | −6.60891 | 0.33723 |
| gb:NM_016134.1 | −6.50573 | 0.48568 |
| gb:M25915.1 | −6.47259 | 0.47914 |
| gb:NM_001148.2 | −6.37227 | 0.43306 |
| gb:AB011126.1 | −6.27707 | 0.42141 |
| gb:H10545 | −6.09142 | 0.46319 |
| gb:AI693516 | −5.92741 | 0.12202 |
| gb:AA563621 | −5.91289 | 0.30769 |
| gb:AF098518.1 | −5.89271 | 0.30966 |
| gb:BG546884 | −5.86072 | 0.49466 |
| gb:NM_004126.1 | −5.83105 | 0.43125 |
| gb:AF055585.1 | −5.68892 | 0.27012 |
| gb:AI583530 | −5.6829 | 0.29813 |
| gb:AA156022 | −5.64983 | 0.41219 |
| gb:AV699353 | −5.64917 | 0.44109 |
| gb:AV646335 | −5.62094 | 0.41086 |
| gb:AA551075 | −5.59766 | 0.48937 |
| gb:M25915.1 | −5.5744 | 0.47603 |
| gb:NM_020482.1 | −5.51417 | 0.39657 |
| gb:AF162704.1 | −5.48088 | 0.35012 |
| gb:AA461490 | −5.44818 | 0.48097 |
| gb:AI355848 | −5.44226 | 0.40774 |
| gb:NM_000950.1 | −5.43128 | 0.48705 |
| gb:AW022607 | −5.42654 | 0.29281 |
| gb:NM_005822.1 | −5.39812 | 0.15399 |
| gb:AW273796 | −5.35623 | 0.42095 |
| gb:AF044221.1 | −5.3547 | 0.44922 |
| gb:BG035116 | −5.26268 | 0.45706 |
| gb:NM_007168.1 | −5.25994 | 0.19808 |
| gb:BC005916.1 | −5.24978 | 0.38006 |
| gb:NM_002101.2 | −5.21842 | 0.48138 |
| gb:AI380298 | −5.21288 | 0.47699 |
| gb:NM_004657.1 | −5.18841 | 0.33613 |
| gb:NM_003243.1 | −5.18168 | 0.27686 |
| gb:AI422211 | −5.16096 | 0.48293 |
| gb:AA419275 | −5.15374 | 0.38529 |
| gb:NM_003991.1 | −5.12821 | 0.34649 |
| gb:BF195118 | −5.12571 | 0.16280 |
| gb:AA534817 | −5.07192 | 0.16670 |
| gb:BG573647 | −5.07015 | 0.39936 |
| gb:AK023795.1 | −5.06553 | 0.26384 |
| gb:CA447177 | −5.05981 | 0.47887 |
| gb:NM_000019.1 | −5.04263 | 0.42817 |
| gb:AW000995 | −5.04092 | 0.48805 |
| gb:NM_001647.1 | −5.01086 | 0.32470 |
| gb:NM_006867.1 | −5.00905 | 0.48097 |
| gb:M73069.1 | −4.99324 | 0.42923 |
| gb:AI274095 | −4.98036 | 0.14250 |
| gb:AI535735 | −4.97883 | 0.42318 |
| gb:NM_000848.1 | −4.97234 | 0.38225 |
| gb:NM_000430.2 | −4.97123 | 0.41422 |
| gb:BF591534 | −4.96473 | 0.48638 |
| gb:AL551046 | −4.94957 | 0.21635 |
| gb:NM_000385.2 | −4.90068 | 0.35507 |
| gb:BU069195 | −4.89108 | 0.47162 |
| gb:M25813.1 | −4.88695 | 0.24128 |
| gb:AA716107 | −4.87907 | 0.24034 |
| gb:BE044614 | −4.86347 | 0.22412 |
| gb:AF208502.1 | −4.86155 | 0.24113 |
| gb:N22468 | −4.84069 | 0.34234 |
| gb:NM_001451.1 | −4.84008 | 0.31974 |
| gb:AI074333 | −4.83213 | 0.47051 |
| gb:AA489100 | −4.82315 | 0.44476 |
| gb:NM_000898.1 | −4.82301 | 0.26537 |
| gb:AW009747 | −4.80833 | 0.19755 |
| gb:AA524250 | −4.80402 | 0.47515 |
| gb:AF329839.1 | −4.78052 | 0.44541 |
| gb:AI982754 | −4.7773 | 0.42161 |
| gb:AF026219.1 | −4.77691 | 0.47695 |
| gb:W73890 | −4.77309 | 0.33982 |
| gb:BF056892 | −4.75895 | 0.49955 |
| gb:NM_000163.1 | −4.75252 | 0.37152 |
| gb:BF511315 | −4.73049 | 0.42555 |
| gb:AK024784.1 | −4.724 | 0.46003 |
| gb:AV653290 | −4.70208 | 0.49631 |
| gb:AI658662 | −4.66634 | 0.31183 |
| gb:AW130536 | −4.6549 | 0.42992 |
| gb:AW242916 | −4.64557 | 0.45120 |
| gb:AA181256 | −4.63924 | 0.31027 |
| gb:NM_006680.1 | −4.62302 | 0.47044 |
| gb:AI733027 | −4.62007 | 0.45820 |
| gb:BC016647.1 | −4.60594 | 0.47537 |
| gb:BF511231 | −4.60445 | 0.37411 |
| gb:BG253119 | −4.60176 | 0.39866 |
| gb:BE218803 | −4.59858 | 0.28871 |
| gb:AB056476.1 | −4.59321 | 0.33287 |
| gb:NM_024756.1 | −4.59168 | 0.33372 |
| gb:AW192692 | −4.58548 | 0.23358 |
| gb:N53248 | −4.58178 | 0.44727 |
| gb:AA296657 | −4.5569 | 0.43379 |
| gb:BE897886 | −4.5443 | 0.42513 |
| gb:NM_007351.1 | −4.5372 | 0.28290 |
| gb:NM_001386.1 | −4.53114 | 0.47020 |
| gb:NM_019105.1 | −4.51445 | 0.23203 |
| gb:NM_007116.1 | −4.51282 | 0.25676 |
| gb:AI183997 | −4.50201 | 0.37678 |
| gb:NM_000561.1 | −4.50079 | 0.42924 |
| gb:BE250348 | −4.49807 | 0.48021 |
| gb:AF039698.1 | −4.49384 | 0.45601 |
| gb:BC015940.1 | −4.48514 | 0.46118 |
| gb:NM_020163.1 | −4.48207 | 0.41371 |
| gb:AL589603 | −4.46984 | 0.29272 |
| gb:AK026776.1 | −4.46905 | 0.49254 |
| gb:AU126086 | −4.44637 | 0.30975 |
| gb:BE048514 | −4.4383 | 0.37951 |
| gb:NM_024863.1 | −4.4377 | 0.37829 |
| gb:AK054607.1 | −4.43579 | 0.49598 |
| gb:NM_022870.1 | −4.41429 | 0.25529 |
| gb:AI052536 | −4.40948 | 0.33998 |
| gb:S67238.1 | −4.4062 | 0.37548 |
| gb:AF021834.1 | −4.4005 | 0.46148 |
| gb:NM_001290.1 | −4.38816 | 0.27918 |
| gb:NM_003500.1 | −4.37682 | 0.40200 |
| gb:NM_006829.1 | −4.37098 | 0.49343 |
| gb:AL046979 | −4.36917 | 0.39863 |
| gb:AF325503.1 | −4.36754 | 0.18613 |
| gb:BG435302 | −4.36176 | 0.47873 |
| gb:NM_052885.1 | −4.32521 | 0.47388 |
| gb:AB029026.1 | −4.30799 | 0.31061 |
| gb:NM_021069.1 | −4.30538 | 0.18818 |
| gb:AL562152 | −4.30453 | 0.45540 |
| gb:L03203.1 | −4.30028 | 0.38039 |
| gb:BC014479.1 | −4.29415 | 0.41044 |
| gb:NM_016608.1 | −4.27664 | 0.41053 |
| gb:AB028998.1 | −4.27613 | 0.39871 |
| gb:BE467261 | −4.27468 | 0.49455 |
| gb:AA243659 | −4.26975 | 0.46088 |
| gb:AI718937 | −4.26552 | 0.35710 |
| gb:AW138143 | −4.25186 | 0.26262 |
| gb:NM_012436.1 | −4.24705 | 0.43726 |
| gb:AI420144 | −4.23259 | 0.48521 |
| gb:D84109.1 | −4.2202 | 0.45258 |
| gb:AI744499 | −4.21809 | 0.49482 |
| gb:W19983 | −4.20289 | 0.47031 |
| gb:BE879367 | −4.20053 | 0.46487 |
| gb:D84109.1 | −4.19637 | 0.45883 |
| gb:AI479176 | −4.1725 | 0.3381 |
| gb:BF511276 | −4.1712 | 0.49085 |
| gb:AI979276 | −4.15733 | 0.44880 |
| gb:AI150641 | −4.15667 | 0.41388 |
| gb:NM_005424.1 | −4.15633 | 0.32626 |
| gb:D79994.1 | −4.12219 | 0.34907 |
| gb:AL573851 | −4.11101 | 0.41532 |
| gb:BF434995 | −4.10883 | 0.38192 |
| gb:BC025770.1 | −4.0958 | 0.33505 |
| gb:U70862.1 | −4.07626 | 0.46921 |
| gb:NM_005031.2 | −4.0752 | 0.25725 |
| gb:AF493929.1 | −4.06844 | 0.49574 |
| gb:AF207547.1 | −4.06571 | 0.43160 |
| gb:NM_012193.1 | −4.05989 | 0.43609 |
| gb:AI439556 | −4.04641 | 0.47849 |
| gb:NM_030820.1 | −4.04317 | 0.44912 |
| gb:AA628586 | −4.03627 | 0.23043 |
| gb:NM_015493.1 | −4.03334 | 0.37273 |
| gb:AU152178 | −4.02866 | 0.37548 |
| gb:NM_006097.1 | −4.02712 | 0.37586 |
| gb:AW269340 | −4.02586 | 0.47052 |
| gb:AW500180 | −4.01742 | 0.44026 |
| gb:NM_000609.1 | −4.01456 | 0.39640 |
| gb:H47630 | −4.00451 | 0.42120 |
| gb:AL577322 | −3.9897 | 0.41823 |
| gb:H15920 | −3.96637 | 0.42780 |
| gb:M64497.1 | −3.95384 | 0.42365 |
| gb:BE856637 | −3.93729 | 0.48506 |
| gb:AI634580 | −3.93726 | 0.18132 |
| gb:AB011110.2 | −3.93505 | 0.32885 |
| gb:NM_006283.1 | −3.93057 | 0.44123 |
| gb:BF724137 | −3.91477 | 0.30967 |
| gb:AF043290.1 | −3.90639 | 0.44403 |
| gb:NM_001937.2 | −3.89812 | 0.47010 |
| gb:NM_007106.1 | −3.89477 | 0.40635 |
| gb:NM_002588.1 | −3.87917 | 0.48053 |
| gb:BC001283.1 | −3.87298 | 0.37799 |
| gb:BM546261 | −3.86306 | 0.47818 |
| gb:W61005 | −3.86228 | 0.35186 |
| gb:AI811298 | −3.86154 | 0.44683 |
| gb:NM_005019.1 | −3.85849 | 0.42228 |
| gb:AL117468.1 | −3.85767 | 0.42710 |
| gb:NM_016245.1 | −3.85 | 0.41719 |
| gb:BC002449.1 | −3.84398 | 0.19183 |
| gb:AI799915 | −3.82502 | 0.40132 |
| gb:NM_030817.1 | −3.81721 | 0.42693 |
| gb:BC004287.1 | −3.81667 | 0.46413 |
| gb:NM_021785.2 | −3.81387 | 0.27248 |
| gb:NM_005780.1 | −3.80462 | 0.45746 |
| gb:NM_018094.1 | −3.79887 | 0.49404 |
| gb:AL136550.1 | −3.79304 | 0.22484 |
| gb:NM_003012.2 | −3.79181 | 0.32569 |
| gb:AW298235 | −3.79156 | 0.45538 |
| gb:AI810244 | −3.7866 | 0.49622 |
| gb:N21458 | −3.78561 | 0.45202 |
| gb:AI679268 | −3.78532 | 0.38707 |
| gb:BF941499 | −3.78503 | 0.37677 |
| gb:AI692523 | −3.77409 | 0.24764 |
| gb:H12280 | −3.76729 | 0.49777 |
| gb:BG530481 | −3.76523 | 0.48185 |
| gb:AF226044.1 | −3.74769 | 0.42239 |
| gb:AW469790 | −3.7455 | 0.34705 |
| gb:NM_003078.1 | −3.74116 | 0.46429 |
| gb:AW002079 | −3.72703 | 0.44252 |
| gb:NM_030802.1 | −3.72164 | 0.41762 |
| gb:NM_006729.1 | −3.71728 | 0.48442 |
| gb:AA573140 | −3.70983 | 0.40330 |
| gb:NM_005965.1 | −3.70658 | 0.44543 |
| gb:AU145805 | −3.70391 | 0.44828 |
| gb:AW338933 | −3.69937 | 0.43737 |
| gb:NM_003570.1 | −3.69364 | 0.44332 |
| gb:NM_000362.2 | −3.69215 | 0.44558 |
| gb:NM_020190.1 | −3.68647 | 0.38770 |
| gb:M60485.1 | −3.66646 | 0.44316 |
| gb:NM_001541.1 | −3.65895 | 0.37204 |
| gb:NM_022844.1 | −3.65409 | 0.25973 |
| gb:N95226 | −3.64074 | 0.44039 |
| gb:BF576053 | −3.63777 | 0.43262 |
| gb:NM_002912.1 | −3.6362 | 0.43658 |
| gb:NM_016588.1 | −3.63409 | 0.29575 |
| gb:NM_003793.2 | −3.63221 | 0.46185 |
| gb:AU144912 | −3.6198 | 0.40595 |
| gb:BG540494 | −3.61206 | 0.39631 |
| gb:AW138159 | −3.6079 | 0.41098 |
| gb:AU145127 | −3.60642 | 0.40947 |
| gb:AI635774 | −3.60578 | 0.27564 |
| gb:H28667 | −3.60023 | 0.38828 |
| gb:BC006439.1 | −3.59778 | 0.48721 |
| gb:AF109161.1 | −3.59029 | 0.36754 |
| gb:AL137566.1 | −3.58985 | 0.12211 |
| gb:AI335267 | −3.57991 | 0.44547 |
| gb:NM_002725.1 | −3.5757 | 0.44259 |
| gb:AA722069 | −3.57142 | 0.27875 |
| gb:NM_014880.1 | −3.56059 | 0.28258 |
| gb:AL536517 | −3.54517 | 0.37161 |
| gb:NM_006329.1 | −3.54392 | 0.37516 |
| Cluster Incl. | −3.539 | 0.34838 |
| gb:AL049265.1 | −3.53487 | 0.43104 |
| gb:AI589594 | −3.52292 | 0.45465 |
| gb:AV725664 | −3.51386 | 0.28097 |
| gb:AA149644 | −3.49903 | 0.48479 |
| gb:BF572868 | −3.49756 | 0.45136 |
| gb:BF508662 | −3.49272 | 0.39359 |
| gb:NM_030569.1 | −3.49078 | 0.42305 |
| gb:R44979 | −3.47589 | 0.46375 |
| gb:AK093340.1 | −3.47491 | 0.48803 |
| gb:AI261455 | −3.46652 | 0.17265 |
| gb:NM_004808.1 | −3.46641 | 0.47598 |
| gb:NM_004615.1 | −3.45536 | 0.31950 |
| gb:AW976035 | −3.45143 | 0.27471 |
| Cluster Incl. | −3.45106 | 0.32948 |
| gb:NM_015185.1 | −3.4487 | 0.38224 |
| gb:BG339050 | −3.44328 | 0.40815 |
| gb:AF007150.1 | −3.43434 | 0.45672 |
| gb:AA701657 | −3.43166 | 0.26612 |
| gb:NM_001299.1 | −3.4316 | 0.23776 |
| gb:NM_004982.1 | −3.4282 | 0.45584 |
| gb:AF086641 | −3.42465 | 0.49427 |
| gb:AI806322 | −3.42398 | 0.49041 |
| gb:S81545.1 | −3.41273 | 0.43442 |
| gb:M17252.1 | −3.41212 | 0.32546 |
| gb:AI719730 | −3.40167 | 0.39531 |
| gb:AL565812 | −3.40016 | 0.49949 |
| gb:AA745971 | −3.39405 | 0.46385 |
| gb:AI475164 | −3.39266 | 0.45715 |
| gb:NM_004105.2 | −3.38538 | 0.46272 |
| gb:BC004958.1 | −3.38525 | 0.26385 |
| gb:AI862120 | −3.38244 | 0.20138 |
| gb:NM_014800.1 | −3.36628 | 0.46472 |
| gb:BC041391.1 | −3.36154 | 0.46150 |
| gb:AI817708 | −3.35819 | 0.39635 |
| gb:NM_002023.2 | −3.35734 | 0.48056 |
| gb:BE674089 | −3.35509 | 0.43814 |
| gb:AI281593 | −3.34841 | 0.32336 |
| gb:BC004912.1 | −3.33291 | 0.33545 |
| gb:BF967998 | −3.32676 | 0.34452 |
| gb:AA126505 | −3.32492 | 0.17655 |
| gb:AA909044 | −3.32294 | 0.39601 |
| gb:AI458003 | −3.30556 | 0.31615 |
| gb:AW264082 | −3.30403 | 0.32701 |
| gb:AL569804 | −3.30348 | 0.30641 |
| gb:NM_000627.1 | −3.30153 | 0.45669 |
| gb:M29277.1 | −3.28412 | 0.48282 |
| gb:AI186739 | −3.27391 | 0.38141 |
| gb:NM_016164.2 | −3.26776 | 0.33452 |
| gb:NM_014668.1 | −3.2627 | 0.37378 |
| gb:BE672313 | −3.26124 | 0.47514 |
| gb:AA046411 | −3.25982 | 0.47125 |
| gb:BG285881 | −3.25973 | 0.33529 |
| gb:NM_006102.1 | −3.25538 | 0.44748 |
| gb:AF134802.1 | −3.25004 | 0.38836 |
| gb:BE670797 | −3.24904 | 0.34076 |
| gb:NM_006079.1 | −3.24769 | 0.44246 |
| gb:AL136693.1 | −3.24719 | 0.40222 |
| gb:NM_001864.1 | −3.23727 | 0.44857 |
| gb:BF969970 | −3.23082 | 0.4785 |
| gb:AI701430 | −3.2274 | 0.49535 |
| gb:NM_005544.1 | −3.22709 | 0.45164 |
| gb:NM_016348.1 | −3.22572 | 0.31739 |
| gb:AW469573 | −3.22184 | 0.39798 |
| gb:AI742838 | −3.22164 | 0.49000 |
| gb:BF222893 | −3.21956 | 0.42585 |
| gb:AA541622 | −3.21731 | 0.18242 |
| gb:BE218803 | −3.21598 | 0.43146 |
| gb:AI146848 | −3.21557 | 0.29237 |
| gb:NM_003278.1 | −3.21309 | 0.45799 |
| gb:AV726166 | −3.20308 | 0.44634 |
| gb:BC001755.1 | −3.20261 | 0.24073 |
| gb:NM_007066.1 | −3.20182 | 0.34495 |
| gb:NM_002222.1 | −3.19562 | 0.27120 |
| gb:AL046979 | −3.19496 | 0.22445 |
| gb:NM_000552.2 | −3.19404 | 0.33847 |
| gb:NM_012134.1 | −3.194 | 0.22944 |
| gb:AW189885 | −3.17853 | 0.44806 |
| gb:AI761728 | −3.17796 | 0.42674 |
| gb:NM_001678.1 | −3.17221 | 0.42269 |
| gb:AW269335 | −3.1703 | 0.42790 |
| gb:U69546.1 | −3.16074 | 0.35216 |
| gb:U91903.1 | −3.15926 | 0.46294 |
| gb:BF984227 | −3.15505 | 0.41117 |
| gb:NM_023926.1 | −3.15102 | 0.39985 |
| gb:NM_003165.1 | −3.14767 | 0.42762 |
| gb:NM_001927.1 | −3.1435 | 0.38215 |
| gb:BF591692 | −3.13857 | 0.22352 |
| gb:AI193623 | −3.13555 | 0.40341 |
| gb:AI963304 | −3.12661 | 0.27805 |
| gb:AF324888.1 | −3.1224 | 0.20035 |
| gb:N29918 | −3.12166 | 0.35308 |
| gb:AA889653 | −3.11529 | 0.46107 |
| gb:BG401568 | −3.11228 | 0.38245 |
| gb:AV648364 | −3.11089 | 0.43800 |
| gb:M24317.1 | −3.09775 | 0.13870 |
| gb:BF433341 | −3.09216 | 0.36663 |
| gb:NM_002036.1 | −3.09143 | 0.32325 |
| gb:NM_003829.1 | −3.09073 | 0.35814 |
| gb:AA042983 | −3.08619 | 0.49967 |
| Cluster Incl. | −3.08544 | 0.40813 |
| gb:AF159570.1 | −3.08388 | 0.33374 |
| gb:AL136562.1 | −3.08028 | 0.43941 |
| gb:AA706788 | −3.07641 | 0.17639 |
| gb:NM_020379.1 | −3.0743 | 0.47751 |
| gb:AW236803 | −3.07221 | 0.22043 |
| gb:AL527331 | −3.07197 | 0.47655 |
| gb:N66571 | −3.06954 | 0.42897 |
| gb:AF070621.1 | −3.06944 | 0.29954 |
| gb:NM_003919.1 | −3.06227 | 0.46210 |
| gb:NM_015559.1 | −3.06086 | 0.43994 |
| gb:BF514158 | −3.05812 | 0.42932 |
| gb:AF017987.1 | −3.05705 | 0.44798 |
| gb:NM_000860.1 | −3.05427 | 0.44945 |
| Cluster Incl. | −3.04877 | 0.45843 |
| gb:NM_002667.1 | −3.0353 | 0.21197 |
| gb:BF670447 | −3.03479 | 0.48255 |
| gb:AI951185 | −3.0315 | 0.30523 |
| gb:AI806174 | −3.03115 | 0.41958 |
| gb:AL391688 | −3.02029 | 0.43629 |
| gb:NM_004078.1 | −3.01955 | 0.41837 |
| gb:NM_002753.1 | −3.01662 | 0.48623 |
| gb:NM_002616.1 | −3.01394 | 0.4519 |
| gb:AL050154.1 | −3.00474 | 0.46938 |
| gb:AI801777 | −3.00268 | 0.43901 |
| gb:NM_002526.1 | −2.99627 | 0.43784 |
| gb:AI671186 | −2.99589 | 0.34472 |
| gb:BE673665 | −2.99548 | 0.35426 |
| gb:AW138886 | −2.99474 | 0.46056 |
| gb:AW204712 | −2.99297 | 0.41013 |
| gb:AI828648 | −2.99038 | 0.38637 |
| gb:NM_007203.1 | −2.99028 | 0.42459 |
| gb:NM_021963.1 | −2.98307 | 0.48313 |
| gb:NM_005012.1 | −2.98198 | 0.45676 |
| gb:AW467136 | −2.97755 | 0.43203 |
| gb:AA626780 | −2.9764 | 0.44143 |
| gb:BQ894022 | −2.96896 | 0.47225 |
| gb:NM_002585.1 | −2.96482 | 0.47130 |
| gb:AF225986.1 | −2.96399 | 0.43713 |
| gb:AI417267 | −2.95409 | 0.30079 |
| gb:BC029442.1 | −2.94915 | 0.47380 |
| gb:AB015706.1 | −2.94445 | 0.41564 |
| gb:AB033832.1 | −2.93609 | 0.46305 |
| gb:W94001 | −2.93059 | 0.19190 |
| gb:AA114166 | −2.92491 | 0.47467 |
| gb:AI056877 | −2.92271 | 0.45633 |
| gb:AI969945 | −2.92085 | 0.37157 |
| gb:BF114870 | −2.92012 | 0.39727 |
| gb:AI373166 | −2.9157 | 0.48780 |
| gb:AI927486 | −2.91472 | 0.41655 |
| gb:T68445 | −2.91427 | 0.30601 |
| gb:NM_003916.1 | −2.90653 | 0.38920 |
| gb:NM_001615.2 | −2.90347 | 0.37294 |
| gb:BC021723.1 | −2.89303 | 0.24481 |
| gb:NM_004434.1 | −2.88303 | 0.48852 |
| gb:AA847654 | −2.87807 | 0.44568 |
| gb:AF208967.1 | −2.86874 | 0.42755 |
| gb:AI743596 | −2.86858 | 0.49075 |
| gb:AI972496 | −2.84964 | 0.33191 |
| gb:NM_000248.1 | −2.83277 | 0.37978 |
| gb:AU147419 | −2.83099 | 0.37634 |
| gb:AW069181 | −2.82838 | 0.37925 |
| gb:AI093572 | −2.82452 | 0.46943 |
| gb:N90870 | −2.81943 | 0.28595 |
| gb:NM_006932.1 | −2.81588 | 0.41718 |
| gb:U35139.1 | −2.81559 | 0.38795 |
| gb:AL042523 | −2.80447 | 0.49231 |
| gb:W73230 | −2.80258 | 0.26813 |
| gb:NM_003877.1 | −2.80038 | 0.34301 |
| gb:AI459140 | −2.7963 | 0.40112 |
| gb:AL023553 | −2.79613 | 0.45145 |
| gb:AB046692.1 | −2.79329 | 0.32680 |
| gb:AI141603 | −2.78984 | 0.49467 |
| gb:NM_006006.1 | −2.78538 | 0.48420 |
| gb:T89120 | −2.78114 | 0.48393 |
| gb:AI123555 | −2.77587 | 0.37980 |
| gb:NM_023037.1 | −2.77352 | 0.49518 |
| gb:AW276078 | −2.77112 | 0.38631 |
| gb:AA923372 | −2.7684 | 0.37431 |
| gb:AB020707.1 | −2.76513 | 0.48830 |
| gb:NM_002184.1 | −2.76132 | 0.47446 |
| gb:AI093327 | −2.75291 | 0.48938 |
| gb:NM_006182.1 | −2.74397 | 0.48600 |
| gb:AB056106.1 | −2.74161 | 0.32959 |
| gb:AL042588 | −2.73705 | 0.37629 |
| gb:AI569787 | −2.7319 | 0.29650 |
| gb:AI797276 | −2.73136 | 0.42882 |
| gb:NM_015385.1 | −2.72827 | 0.19116 |
| gb:NM_003022.1 | −2.72656 | 0.44284 |
| gb:U63041.1 | −2.72369 | 0.35422 |
| gb:AF098951.2 | −2.71548 | 0.37566 |
| gb:NM_032817.1 | −2.71377 | 0.48656 |
| gb:BE966768 | −2.70819 | 0.48765 |
| gb:AI471375 | −2.70636 | 0.43326 |
| gb:AB029026.1 | −2.70265 | 0.47483 |
| gb:BF726212 | −2.69959 | 0.29219 |
| gb:AI742570 | −2.69904 | 0.42411 |
| gb:NM_145023.1 | −2.69397 | 0.46467 |
| gb:AL356755 | −2.69262 | 0.30140 |
| Cluster Incl. | −2.68829 | 0.47128 |
| gb:AL049798 | −2.68631 | 0.32190 |
| gb:AI343000 | −2.68458 | 0.29194 |
| gb:NM_003062.1 | −2.6804 | 0.39391 |
| gb:NM_012317.1 | −2.67637 | 0.46943 |
| gb:AA480858 | −2.67493 | 0.39981 |
| gb:BE348466 | −2.67063 | 0.42164 |
| gb:AL574194 | −2.66272 | 0.34799 |
| gb:NM_022003.1 | −2.64653 | 0.46279 |
| gb:AV725825 | −2.64248 | 0.40374 |
| gb:BF056275 | −2.64033 | 0.48955 |
| gb:BG251521 | −2.61546 | 0.41824 |
| gb:AB007877.1 | −2.61402 | 0.47649 |
| gb:R79120 | −2.60388 | 0.45960 |
| gb:BG163478 | −2.60234 | 0.42410 |
| gb:Z24725.1 | −2.60179 | 0.32877 |
| gb:BE882538 | −2.59956 | 0.37255 |
| gb:BC010946.1 | −2.5948 | 0.44692 |
| gb:AI672386 | −2.5907 | 0.25256 |
| gb:AV733950 | −2.58693 | 0.49034 |
| gb:BG289306 | −2.57879 | 0.29359 |
| gb:AI129320 | −2.57383 | 0.40386 |
| gb:NM_002084.2 | −2.57217 | 0.48144 |
| gb:AA401248 | −2.57044 | 0.43174 |
| gb:AW271199 | −2.56784 | 0.37418 |
| gb:BF793701 | −2.5583 | 0.38931 |
| gb:AI733330 | −2.55155 | 0.47014 |
| gb:AL117653.1 | −2.54479 | 0.40154 |
| gb:AK026829.1 | −2.54259 | 0.47618 |
| gb:N26005 | −2.535 | 0.28652 |
| gb:Y12885.1 | −2.53251 | 0.38652 |
| gb:AL048386 | −2.52991 | 0.41214 |
| gb:AA131324 | −2.52955 | 0.48166 |
| gb:AW149846 | −2.51439 | 0.46609 |
| gb:R30807 | −2.51071 | 0.48421 |
| gb:BF510533 | −2.51022 | 0.39587 |
| gb:AL047908 | −2.50491 | 0.48357 |
| gb:AL022726 | −2.50308 | 0.48432 |
| gb:AB007830.1 | −2.50002 | 0.41676 |
| gb:NM_001232.1 | −2.49611 | 0.39469 |
| gb:AI929792 | −2.48362 | 0.49025 |
| gb:AI803181 | −2.48143 | 0.30086 |
| gb:AI754416 | −2.47497 | 0.36023 |
| http://genome- | −2.46647 | 0.28356 |
| gb:AL037805 | −2.46156 | 0.39456 |
| gb:U61536.1 | −2.46154 | 0.36106 |
| gb:X79990.1 | −2.45973 | 0.32246 |
| gb:NM_001400.2 | −2.45803 | 0.46877 |
| gb:NM_001463.1 | −2.45598 | 0.49076 |
| gb:AI638433 | −2.45234 | 0.2871 |
| gb:AL162032.1 | −2.44917 | 0.44044 |
| gb:AA480677 | −2.4458 | 0.41464 |
| gb:BF732712 | −2.43923 | 0.46814 |
| gb:AI096375 | −2.43911 | 0.43412 |
| gb:M21692.1 | −2.43258 | 0.33262 |
| gb:BF697865 | −2.42438 | 0.44259 |
| gb:AF095192.1 | −2.42254 | 0.47605 |
| gb:AV700059 | −2.42233 | 0.30272 |
| gb:NM_000961.1 | −2.4175 | 0.44751 |
| gb:AW193693 | −2.41542 | 0.48165 |
| gb:AF007162.1 | −2.4063 | 0.47912 |
| gb:D38300.1 | −2.40299 | 0.46744 |
| gb:NM_000587.1 | −2.39141 | 0.22409 |
| gb:D50683.1 | −2.38494 | 0.43496 |
| gb:BC001830.1 | −2.3836 | 0.49790 |
| gb:AI086614 | −2.3809 | 0.43218 |
| gb:AF141347.1 | −2.38049 | 0.44766 |
| gb:AA430014 | −2.3798 | 0.41441 |
| gb:BC029855.1 | −2.37751 | 0.45745 |
| gb:AL523076 | −2.36826 | 0.39135 |
| gb:AV706522 | −2.361 | 0.41603 |
| gb:BC040053.1 | −2.35584 | 0.45617 |
| gb:NM_000055.1 | −2.35079 | 0.40426 |
| gb:AI798790 | −2.34652 | 0.34091 |
| gb:AA868896 | −2.32354 | 0.49969 |
| gb:NM_017734.1 | −2.318 | 0.48434 |
| gb:NM_002612.1 | −2.31172 | 0.40725 |
| gb:AL703532 | −2.31073 | 0.29996 |
| gb:NM_003507.1 | −2.30679 | 0.42540 |
| gb:NM_001911.1 | −2.30493 | 0.47502 |
| gb:NM_014890.1 | −2.30249 | 0.42990 |
| gb:D38298.1 | −2.30134 | 0.42171 |
| gb:L27489.1 | −2.30026 | 0.48167 |
| gb:AL050152.1 | −2.29829 | 0.27537 |
| gb:AL042088 | −2.29539 | 0.36291 |
| gb:AW206234 | −2.29315 | 0.49382 |
| gb:NM_024426.1 | −2.29113 | 0.43931 |
| gb:AB004903.1 | −2.28717 | 0.43814 |
| gb:AW138143 | −2.27853 | 0.18413 |
| gb:BC000686.1 | −2.26904 | 0.45338 |
| gb:AL049381.1 | −2.26543 | 0.24683 |
| gb:BG112263 | −2.26539 | 0.49487 |
| gb:AI741439 | −2.26196 | 0.47846 |
| gb:AI377043 | −2.25635 | 0.45772 |
| gb:AI674243 | −2.25596 | 0.46848 |
| gb:NM_017637.1 | −2.24989 | 0.42737 |
| gb:NM_025226.1 | −2.24268 | 0.39761 |
| gb:AI086864 | −2.23363 | 0.43289 |
| gb:AA093668 | −2.23356 | 0.47325 |
| gb:NM_014424.1 | −2.2327 | 0.21898 |
| gb:AI290475 | −2.23151 | 0.48112 |
| gb:AL049176 | −2.23147 | 0.38687 |
| gb:AA551090 | −2.23013 | 0.38771 |
| gb:U23850.1 | −2.22979 | 0.42355 |
| gb:BG492376 | −2.22012 | 0.35974 |
| gb:AI680541 | −2.21015 | 0.45895 |
| gb:BG165333 | −2.19856 | 0.46740 |
| gb:NM_002667.1 | −2.19822 | 0.41018 |
| gb:AL037401 | −2.19711 | 0.47960 |
| gb:AA994712 | −2.19457 | 0.46877 |
| gb:M63889.1 | −2.1943 | 0.39286 |
| gb:NM_016563.1 | −2.18986 | 0.33253 |
| gb:AA526844 | −2.18463 | 0.44066 |
| gb:W93705 | −2.17991 | 0.29929 |
| gb:AU145682 | −2.17035 | 0.47130 |
| gb:NM_005715.1 | −2.16358 | 0.49618 |
| gb:AB002365.1 | −2.16204 | 0.35188 |
| gb:AB015228.1 | −2.16037 | 0.43863 |
| gb:AW157094 | −2.15899 | 0.35578 |
| gb:AL137578.1 | −2.15458 | 0.41185 |
| gb:NM_002837.1 | −2.15203 | 0.45563 |
| gb:BF449063 | −2.15057 | 0.23344 |

What is claimed is:

1. A method for the diagnosis or staging of cervical cancer, the method comprising:
determining the upregulation of expression of a genetic sequence selected from those listed in Table 2, groups I and Group II, Table 5 and/or Table 6.

2. The method according to claim 1, wherein said cervical cancer is squamous cell carcinoma of the cervix.

3. The method according to claim 1, wherein said determining comprises detecting increased or decreased amounts of mRNA or polypeptide in a sample of cervical cells.

4. The method according to claim 1, wherein expression is determined by real time PCR.

5. The method according to claim 4, wherein said PCR is performed with a set of primers set forth in Table 1.

6. The method according to claim 1, wherein expression is determined by hybridization to an array.

7. The method according to claim 6, wherein said array comprises two or more sequences set forth in Table 2, groups I and Group II, Table 5 and/or Table 6.

8. A method of imaging a cervical cancer, the method comprising:
administering to a patient an effective amount of a compound that specifically binds a polypeptide encoded by a genetic sequence set forth in Table 2 and/or Table 5 wherein said compound is conjugated to an imaging moiety; and
visualizing the imaging moiety of said conjugate.

9. The method of claim 8, wherein said compound is an antibody or antibody fragment.

10. A method of screening candidate agents for modulation of a cervical cancer target protein, the method comprising:
combining a candidate biologically active agent with any one of:
(a) a polypeptide encoded by encoded by a genetic sequence set forth in Table 2, groups I and Group II, Table 5 and/or Table 6;
(b) a cell comprising a nucleic acid encoding and expressing a polypeptide encoded by encoded by a genetic sequence set forth in Table 2, groups I and Group II, Table 5 and/or Table 6; and
determining the effect of said agent on activity of the polypeptide, wherein agents that modulate said polypeptide activity provide a candidate therapeutic agent for cervical cancer.

11. The method according to claim 10, wherein said biologically active agent downregulates expression.

12. The method according to claim 10, wherein said biologically active agent inhibits activity of said polypeptide.
